(19) United States
(12) Patent Application Publication
(10) Pub. No.: US 2006/0094020 A1
(43) Pub. Date: May 4, 2006

(54) Methods and compositions for determining resistance or susceptibility of HIV-1 to stavudine

(76) Inventors: Neil T. Parkin, Belmont, CA (US); Colombe Chappey, San Francisco, CA (US)

Correspondence Address: JONES DAY, 222 EAST 41ST ST, NEW YORK, NY, 10017, US

(21) Appl. No.: 10/976,721

(22) Filed: Oct. 29, 2004

(51) Int. Cl. C12Q 1/70; C12Q 1/68

(52) U.S. Cl. 435/6; 435/5

(57) ABSTRACT

This invention relates to methods for determining resistance or susceptibility of HIV-1 viruses to d4T based on the viral genotypes. The methods generally comprise detecting, in a gene encoding reverse transcriptase of the HIV-1, the presence of a mutation at codon 65 in combination with one or more mutations at certain other codons. Combinations of mutations associated with resistance and susceptibility to d4T are disclosed.

### 1. FIELD OF INVENTION

This invention relates, in part, to methods and compositions for determining resistance or susceptibility of a human immunodeficiency virus (“HIV”) to stavudine (“d4T”) by detecting the presence of combinations of mutations in the gene encoding HIV reverse transcriptase that are associated with resistance or susceptibility to d4T.

### 2. BACKGROUND OF THE INVENTION

More than 60 million people have been infected with the human immunodeficiency virus (“HIV”), the causative agent of acquired immune deficiency syndrome (“AIDS”), since the early 1980s. See Lucas, 2002, Lepr Rev. 73(1):64-71. HIV/AIDS is now the leading cause of death in sub-Saharan Africa, and is the fourth biggest killer worldwide. At the end of 2001, an estimated 40 million people were living with HIV globally. See Norris, 2002, Radiol Technol. 73(4):339-363.

Modem anti-HIV drugs target different stages of the HIV life cycle and a variety of enzymes essential for HIV's replication and/or survival. Amongst the drugs that have so far been approved for AIDS therapy are nucleoside reverse transcriptase inhibitors (“NRTIs”) such as AZT, ddI, ddC, d4T, 3TC, and abacavir; nucleotide reverse transcriptase inhibitors such as tenofovir; non-nucleoside reverse transcriptase inhibitors (“NNRTIs”) such as nevirapine, efavirenz, and delavirdine; protease inhibitors (“PIs”) such as saquinavir, ritonavir, indinavir, nelfinavir, amprenavir, lopinavir and atazanavir; and fusion inhibitors, such as enfuvirtide.

Nonetheless, in the vast majority of subjects none of these antiviral drugs, either alone or in combination, proves effective either to prevent eventual progression of chronic HIV infection to AIDS or to treat acute AIDS. This phenomenon is due, in part, to the high mutation rate of HIV and the rapid emergence of mutant HIV strains that are resistant to antiviral therapeutics upon administration of such drugs to infected individuals. Among such mutant strains are those, for example, that contain the K65R mutation in reverse transcriptase. This mutation is known to correlate with reduced susceptibility to a number of NRTIs, including, for example, didanosine, abacavir, tenofovir, and lamivudine. See, e.g., Wainberg & Turner, 2004, J. Acquir. Immune Defic. Syndr. 37(Suppl 1):S36-S43. However, the effect of K65R on d4T susceptibility remains unclear. Some reports indicate that K65R is selected in the presence of d4T. See, e.g., Garcia-Lerma et al., 2203, J. Virol. 77:5685-5693. However, these reports indicate that K65R alone is not sufficient to provide high-level resistance to d4T. Individual viral isolates selected by passage through cell culture in the presence of d4T exhibit certain combinations of mutations in conjunction with K65R and reduced susceptibility to d4T. See id. However, no reports have identified that the K65R mutation can be selected by d4T in the absence of other antiviral drugs in vivo rather than in cell culture.

Nonetheless, no reports to date have demonstrated that any mutations in HIV reverse transcriptase, in association with K65R, correlate with reduced d4T susceptibility in a statistically significant manner. An analysis that identifies mutations that significantly correlate with d4T resistance or susceptibility in the presence of K65R would be very useful in determining the role of this mutation in d4T resistance and in guiding therapeutic decisions in the treatment of HIV-infected individuals, particularly those infected with HIV that comprises the K65R mutation. Such analysis would also help to elucidate the potential for selection of K65R in vivo by d4T therapy. For the first time, these, as well as other unmet needs, will be achievable as a result of the invention described hereinafter.

### 3. SUMMARY OF THE INVENTION

The present invention provides methods for determining that an HIV-1 is resistant or susceptible to d4T. In the methods, resistance or susceptibility to d4T can be determined by detecting the presence of mutations that correlate with resistance or susceptibility to d4T.

Thus, in certain aspects, the invention provides a method for determining that an HIV-1 is resistant to d4T, comprising detecting in a gene encoding reverse transcriptase of the HIV-1 the presence of a mutation at codon 65 in combination with a mutation in at least one of codon 40, 67, 69, 70, 75, 77, 116, 139, 169, 181, 190, 210, 215, 218, 219, 221, 224, or 227, wherein the presence of the mutations correlates with resistance to d4T, thereby determining that the HIV-1 is resistant to d4T. The presence of the mutations associated with resistance to d4T can be detected according to any method known to one of skill in the art without limitation. Methods for detecting such mutations are described extensively below.

In other aspects, the invention provides a method for determining that an HIV-1 is susceptible to d4T, comprising detecting in a gene encoding reverse transcriptase of the HIV-1 the presence of a mutation at codon 65 in combination with a mutation at codon 135 and/or 184, wherein the presence of said mutations correlates with susceptibility to d4T, thereby determining that the HIV-1 is susceptible to d4T. The presence of the mutations associated with susceptibility to d4T can be detected according to any method known to one of skill in the art without limitation. Methods for detecting such mutations are described extensively below.

### 4. BRIEF DESCRIPTION OF THE FIGURES

FIG. 1 presents the distribution of changes in IC₅₀ to an array of NRTIs identified in 626 individual patient-derived viruses that comprise the K65R mutation in RT, excluding the presence of mutations Q151M and T69ins, which mutations are known to correlate with NRTI resistance, including d4T resistance.

FIG. 2 presents the distribution of changes in IC₅₀ to an array of NRTIs identified in 202 individual patient-derived viruses that comprise the K65R mutation in RT alone.

FIG. 3 presents a table showing mutations in HIV-1 RT that are associated with resistance or susceptibility to d4T using Fisher's Exact Test in combination with K65R.

FIG. 4 presents a regression tree analysis performed using the entropy splitting method of CART 5.0 (Salford Systems; San Diego, Calif.) that diagrams the relative contributions of RT mutations that correlate most strongly with resistance to d4T.

FIG. 5 presents a regression tree analysis performed using the GINI splitting method (Insightful Miner 3.0; Insightfuil Corp, Seattle, Wash.) that diagrams the relative contributions of RT mutations that correlate most strongly with resistance to d4T.

FIG. 6 presents a set of amino acid alignments demonstrating that certain HIV deletion mutants can be assigned to have deletions at D67 or T69; in this analysis, all such deletion mutants when present in combination with S68G are assigned to position 69.

### 5. DETAILED DESCRIPTION OF THE INVENTION

The present invention provides methods for determining that an HIV-1 is resistant or susceptible to antiviral therapy with d4T. The methods generally comprise detecting the presence of mutations in the HIV-1 gene encoding RT that significantly correlate with resistance or susceptibility to d4T.

### 5.1. Abbreviations

“NRTI” is an abbreviation for nucleoside reverse transcriptase inhibitor.

“NNRTI” is an abbreviation for non nucleoside reverse transcriptase inhibitor.

“PI” is an abbreviation for protease inhibitor.

“PR” is an abbreviation for protease.

“RT” is an abbreviation for reverse transcriptase.

“PCR” is an abbreviation for “polymerase chain reaction.”

“HBV” is an abbreviation for hepatitis B virus.

“HCV” is an abbreviation for hepatitis C virus.

“HIV” is an abbreviation for human immunodeficiency virus.

“d4T” is an abbreviation for the NRTI stavudine.

The amino acid notations used herein for the twenty genetically encoded L-amino acids are conventional and are as follows:

| | One-Letter | Three Letter |
|---|---|---|
| Amino Acid | Abbreviation | Abbreviation |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

Unless noted otherwise, when polypeptide sequences are presented as a series of one-letter and/or three-letter abbreviations, the sequences are presented in the N→C direction, in accordance with common practice.

Individual amino acids in a sequence are represented herein as AN, wherein A is the standard one letter symbol for the amino acid in the sequence, and N is the position in the sequence. Mutations are represented herein as A₁NA₂, wherein A₁ is the standard one letter symbol for the amino acid in the reference protein sequence, A₂ is the standard one letter symbol for the amino acid in the mutated protein sequence, and N is the position in the amino acid sequence. For example, a G25M mutation represents a change from glycine to methionine at amino acid position 25. Mutations may also be represented herein as NA₂, wherein N is the position in the amino acid sequence and A₂ is the standard one letter symbol for the amino acid in the mutated protein sequence (e.g., 25M, for a change from the wild-type amino acid to methionine at amino acid position 25). Additionally, mutations may also be represented herein as A₁NX, wherein A₁ is the standard one letter symbol for the amino acid in the reference protein sequence, N is the position in the amino acid sequence, and X indicates that the mutated amino acid can be any amino acid (e.g., G25X represents a change from glycine to any amino acid at amino acid position 25). This notation is typically used when the amino acid in the mutated protein sequence is either not known or, if the amino acid in the mutated protein sequence could be any amino acid, except that found in the reference protein sequence. The amino acid positions are numbered based on the full-length sequence of the protein from which the region encompassing the mutation is derived. Representations of nucleotides and point mutations in DNA sequences are analogous.

The abbreviations used throughout the specification to refer to nucleic acids comprising specific nucleobase sequences are the conventional one-letter abbreviations. Thus, when included in a nucleic acid, the naturally occurring encoding nucleobases are abbreviated as follows: adenine (A), guanine (G), cytosine (C), thymine (T) and uracil (U). Unless specified otherwise, single-stranded nucleic acid sequences that are represented as a series of one-letter abbreviations, and the top strand of double-stranded sequences, are presented in the 5′→3′ direction.

### 5.2. Definitions

As used herein, the following terms shall have the following meanings:

A “phenotypic assay” is a test that measures a phenotype of a particular virus, such as, for example, HIV, or a population of viruses, such as, for example, the population of HIV infecting a subject. The phenotypes that can be measured include, but are not limited to, the resistance or susceptibility of a virus, or of a population of viruses, to a specific anti-viral agent or that measures the replication capacity of a virus.

A “genotypic assay” is an assay that determines a genotype of an organism, a part of an organism, a population of organisms, a gene, a part of a gene, or a population of genes. Typically, a genotypic assay involves determination of the nucleic acid sequence of the relevant gene or genes. Such assays are frequently performed in HIV to establish, for example, whether certain mutations are associated with drug resistance or altered replication capacity are present.

As used herein, “genotypic data” are data about the genotype of, for example, a virus. Examples of genotypic data include, but are not limited to, the nucleotide or amino acid sequence of a virus, a population of viruses, a part of a virus, a viral gene, a part of a viral gene, or the identity of one or more nucleotides or amino acid residues in a viral nucleic acid or protein.

The term “% sequence identity” is used interchangeably herein with the term “% identity” and refers to the level of amino acid sequence identity between two or more peptide sequences or the level of nucleotide sequence identity between two or more nucleotide sequences, when aligned using a sequence alignment program. For example, as used herein, 80% identity means the same thing as 80% sequence identity determined by a defined algorithm, and means that a given sequence is at least 80% identical to another length of another sequence. Exemplary levels of sequence identity include, but are not limited to, 60, 70, 80, 85, 90, 95, 98% or more sequence identity to a given sequence.

The term “% sequence homology” is used interchangeably herein with the term “% homology” and refers to the level of amino acid sequence homology between two or more peptide sequences or the level of nucleotide sequence homology between two or more nucleotide sequences, when aligned using a sequence alignment program. For example, as used herein, 80% homology means the same thing as 80% sequence homology determined by a defined algorithm, and accordingly a homologue of a given sequence has greater than 80% sequence homology over a length of the given sequence. Exemplary levels of sequence homology include, but are not limited to, 60, 70, 80, 85, 90, 95, 98% or more sequence homology to a given sequence.

Exemplary computer programs which can be used to determine identity between two sequences include, but are not limited to, the suite of BLAST programs, e.g., BLASTN, BLASTX, and TBLASTX, BLASTP and TBLASTN, publicly available on the Internet at the NCBI website. See also Altschul et al., 1990, J. Mol. Biol. 215:403-10 (with special reference to the published default setting, i.e., parameters w=4, t=17) and Altschul et al., 1997, Nucleic Acids Res., 25:3389-3402. Sequence searches are typically carried out using the BLASTP program when evaluating a given amino acid sequence relative to amino acid sequences in the GenBank Protein Sequences and other public databases. The BLASTX program is preferred for searching nucleic acid sequences that have been translated in all reading frames against amino acid sequences in the GenBank Protein Sequences and other public databases. Both BLASTP and BLASTX are run using default parameters of an open gap penalty of 11.0, and an extended gap penalty of 1.0, and utilize the BLOSUM-62 matrix. See id.

A preferred alignment of selected sequences in order to determine “% identity” between two or more sequences, is performed using for example, the CLUSTAL-X program, operated with default parameters, including an open gap penalty of 10.0, an extended gap penalty of 0.1, and a BLOSUM 30 similarity matrix.

“Polar Amino Acid” refers to a hydrophilic amino acid having a side chain that is uncharged at physiological pH, but which has at least one bond in which the pair of electrons shared in common by two atoms is held more closely by one of the atoms. Genetically encoded polar amino acids include Asn (N), Gln (Q) Ser (S) and Thr (T).

“Nonpolar Amino Acid” refers to a hydrophobic amino acid having a side chain that is uncharged at physiological pH and which has bonds in which the pair of electrons shared in common by two atoms is generally held equally by each of the two atoms (i.e., the side chain is not polar). Genetically encoded nonpolar amino acids include Ala (A), Gly (G), Ile (I), Leu (L), Met (M) and Val (V).

“Hydrophilic Amino Acid” refers to an amino acid exhibiting a hydrophobicity of less than zero according to the normalized consensus hydrophobicity scale of Eisenberg et al., 1984, J. Mol. Biol. 179:125-142. Genetically encoded hydrophilic amino acids include Arg (R), Asn (N), Asp (D), Glu (E), Gln (Q), His (H), Lys (K), Ser (S) and Thr (T).

“Hydrophobic Amino Acid” refers to an amino acid exhibiting a hydrophobicity of greater than zero according to the normalized consensus hydrophobicity scale of Eisenberg et al., 1984, J. Mol. Biol. 179:125-142. Genetically encoded hydrophobic amino acids include Ala (A), Gly (G), Ile (I), Leu (L), Met (M), Phe (F), Pro (P), Trp (W), Tyr (Y) and Val (V).

“Acidic Amino Acid” refers to a hydrophilic amino acid having a side chain pK value of less than 7. Acidic amino acids typically have negatively charged side chains at physiological pH due to loss of a hydrogen ion. Genetically encoded acidic amino acids include Asp (D) and Glu (E).

“Basic Amino Acid” refers to a hydrophilic amino acid having a side chain pK value of greater than 7. Basic amino acids typically have positively charged side chains at physiological pH due to association with a hydrogen ion. Genetically encoded basic amino acids include Arg (R), His (H) and Lys (K).

A “mutation” is a change in an amino acid sequence or in a corresponding nucleic acid sequence relative to a reference nucleic acid or polypeptide. For embodiments of the invention comprising HIV protease or reverse transcriptase, the reference nucleic acid encoding protease or reverse transcriptase is the protease or reverse transcriptase coding sequence, respectively, present in NL4-3 HIV (GenBank Accession No. AF324493). Likewise, the reference protease or reverse transcriptase polypeptide is that encoded by the NL4-3 HIV sequence. Although the amino acid sequence of a peptide can be determined directly by, for example, Edman degradation or mass spectroscopy, more typically, the amino sequence of a peptide is inferred from the nucleotide sequence of a nucleic acid that encodes the peptide. Any method for determining the sequence of a nucleic acid known in the art can be used, for example, Maxam-Gilbert sequencing (Maxam et al., 1980, Methods in Enzymology 65:499), dideoxy sequencing (Sanger et al., 1977, Proc. Natl. Acad. Sci. USA 74:5463) or hybridization-based approaches (see e.g., Sambrook et al., 2001, Molecular Cloning: A Laboratory Manual, Cold Spring Harbor Laboratory, 3^{rd} ed., NY; and Ausubel et al., 1989, Current Protocols in Molecular Biology, Greene Publishing Associates and Wiley Interscience, NY).

A “mutant” is a virus, gene or protein having a sequence that has one or more changes relative to a reference virus, gene or protein.

The terms “peptide,” “polypeptide” and “protein” are used interchangeably throughout.

The term “wild-type” refers to a viral genotype that does not comprise a mutation known to be associated with drug resistance.

The terms “polynucleotide,” “oligonucleotide” and “nucleic acid” are used interchangeably throughout.

### 5.3. Methods of Determining Resistance or Susceptibility to d4T

In certain aspects, the present invention provides methods for determining that an HIV-1 is resistant or susceptible to the NRTI d4T. In general, the methods comprise detecting the presence of mutations significantly correlated with d4T resistance or susceptibility in the gene encoding reverse transcriptase of the HIV-1, as demonstrated by the examples below.

Therefore, in certain embodiments, the invention provides a method for determining that an HIV-1 is resistant to d4T that comprises detecting in a gene encoding reverse transcriptase of the HIV-1 the presence of a mutation at codon 65 in combination with a mutation at codon 40, 67, 69, 70, 75, 77, 116, 139, 169, 181, 190, 210, 215, 218, 219, 221, 224, or 227, wherein the presence of the mutations correlates with resistance to d4T.

In certain embodiments, the mutation at codon 65 encodes arginine (R).

In certain embodiments, the reverse transcriptase of the HIV-1 comprises a mutation at codon 40. In certain embodiments, the mutation at codon 40 encodes aspartic acid (D) or arginine (R). In certain embodiments, the reverse transcriptase of the HIV-1 comprises a mutation at codon 67. In certain embodiments, the mutation at codon 67 encodes glycine (G), asparagine (N), valine (V), or histidine (H).

In certain embodiments, the reverse transcriptase of the HIV-1 comprises a mutation at codon 69. In certain embodiments, the mutation at codon 69 is a deletion of the amino acid present at codon 69 in a wild-type HIV-1 or encodes alanine (A), aspartic acid (D), isoleucine (I), asparagine (N), or serine (S).

In certain embodiments, the reverse transcriptase of the HIV-1 comprises a mutation at codon 70. In certain embodiments, the mutation at codon 70 encodes cysteine, (C), glycine (G), histidine (H), asparagine (N), arginine (R), or threonine (T).

In certain embodiments, the reverse transcriptase of the HIV-1 comprises a mutation at codon 75. In certain embodiments, the mutation at codon 75 encodes leucine (L), methionine (M), threonine (T), isoleucine (I), or alanine (A). In certain embodiments, the reverse transcriptase of the HIV-1 comprises a mutation at codon 77. In certain embodiments, the mutation at codon 77 encodes leucine (L).

In certain embodiments, the reverse transcriptase of the HIV-1 comprises a mutation at codon 116. In certain embodiments, the mutation at codon 116 encodes proline (P), histidine (H), or tyrosine (Y). In certain embodiments, the reverse transcriptase of the HIV-1 comprises a mutation at codon 139. In certain embodiments, the mutation at codon 139 encodes alanine (A) or threonine (T).

In certain embodiments, the reverse transcriptase of the HIV-1 comprises a mutation at codon 169. In certain embodiments, the mutation at codon 169 encodes alanine (A), aspartic acid (D), or lysine (K). In certain embodiments, the reverse transcriptase of the HIV-1 comprises a mutation at codon 181. In certain embodiments, the mutation at codon 181 encodes cysteine (C), glycine (G), isoleucine (I), leucine (L), or valine (V).

In certain embodiments, the reverse transcriptase of the HIV-1 comprises a mutation at codon 190. In certain embodiments, the mutation at codon 190 encodes alanine (A), cysteine (C), glutamic acid (E), glutamine (Q), serine (S), or valine (V). In certain embodiments, the reverse transcriptase of the HIV-1 comprises a mutation at codon 210. In certain embodiments, the mutation at codon 210 encodes phenylalanine (F), methionine (M), or tryptophan (W).

In certain embodiments, the reverse transcriptase of the HIV-1 comprises a mutation at codon 215. In certain embodiments, the mutation at codon 215 encodes alanine (A), cysteine (C), aspartic acid (D), glutamic acid (E), isoleucine (I), serine (S), valine (V), or tyrosine (Y). In certain embodiments, the reverse transcriptase of the HIV-1 comprises a mutation at codon 218. In certain embodiments, the mutation at codon 218 encodes glutamic acid (E).

In certain embodiments, the reverse transcriptase of the HIV-1 comprises a mutation at codon 219. In certain embodiments, the mutation at codon 219 encodes histidine (H), threonine (T), arginine (R), glutamine (Q), or glutamic acid (E). In certain embodiments, the reverse transcriptase of the HIV-1 comprises a mutation at codon 221. In certain embodiments, the mutation at codon 221 encodes tyrosine (Y).

In certain embodiments, the reverse transcriptase of the HIV-1 comprises a mutation at codon 224. In certain embodiments, the mutation at codon 224 encodes aspartic acid (D). In certain embodiments, the reverse transcriptase of the HIV-1 comprises a mutation at codon 227. In certain embodiments, the mutation at codon 227 encodes leucine (L).

In other embodiments, the method for determining that an HIV-1 is resistant to d4T comprises detecting in a gene encoding reverse transcriptase of the HIV-1 the presence of a mutation at codon 65 in combination with a mutation significantly associated with d4T resistance in the presence of a mutation at codon 65 as indicated by Table 1. In certain embodiments, the significance of the association of the mutation with d4T resistance in the presence of a mutation at codon 65 is indicated by an odds ratio greater than 1. In certain embodiments, the significance of the association of the mutation with d4T resistance in the presence of a mutation at codon 65 is indicated by a p-value calculated in Fisher's Exact Test less than 0.05.

In still other embodiments, the invention provides a method for determining that an HIV-1 is susceptible to d4T that comprises detecting in a gene encoding reverse transcriptase of the HIV-1 the presence of a mutation at codon 65 in combination with a mutation at codon 135 or 184, wherein the presence of the mutations correlates with susceptibility to d4T, thereby determining that the HIV-1 is sensitive to d4T.

In certain embodiments, the reverse transcriptase of the HIV-1 comprises a mutation at codon 135. In certain embodiments, the mutation at codon 135 encodes lysine (K), leucine (L), methionine (M), arginine (R), threonine (T), or valine (V). In certain embodiments, the reverse transcriptase of the HIV-1 comprises a mutation at codon 184. In certain embodiments, the mutation at codon 184 encodes isoleucine (I) or valine (V).

In yet other embodiments, the method for determining that an HIV-1 is susceptible to d4T comprises detecting in a gene encoding reverse transcriptase of the HIV-1 the presence of a mutation at codon 65 in combination with a mutation significantly associated with d4T susceptibility in the presence of a mutation at codon 65 as indicated by Table 1. In certain embodiments, the significance of the association of the mutation with d4T susceptibility in the presence of a mutation at codon 65 is indicated by an odds ratio less than 1. In certain embodiments, the significance of the association of the mutation with d4T susceptibility in the presence of a mutation at codon 65 is indicated by a p-value calculated in Fisher's Exact Test less than 0.05.

### 5.4. Measuring Resistance of HIV-1 to d4T

Any method known in the art can be used to determine a viral drug resistance phenotype, without limitation. See e.g., U.S. Pat. Nos. 5,837,464 and 6,242,187, each of which is hereby incorporated by reference in its entirety.

In certain embodiments, the phenotypic analysis is performed using recombinant virus assays (“RVAs”). RVAs use virus stocks generated by homologous recombination between viral vectors and viral gene sequences, amplified from the patient virus. In certain embodiments, the viral vector is a HIV vector and the viral gene sequences are protease and/or reverse transcriptase and/or gag sequences.

In preferred embodiments, the phenotypic analysis of d4T resistance or susceptibility is performed using PHENOSENSE™ (ViroLogic Inc., South San Francisco, Calif.). See Petropoulos et al., 2000, Antimicrob. Agents Chemother. 44:920-928; U.S. Pat. Nos. 5,837,464 and 6,242,187. PHENOSENSE™ is a phenotypic assay that achieves the benefits of phenotypic testing and overcomes the drawbacks of previous assays. Because the assay has been automated, PHENOSENSE™ provides high throughput methods under controlled conditions for determining replication capacity of a large number of individual viral isolates.

The result is an assay that can quickly and accurately define both the replication capacity and the susceptibility profile of a patient's HIV (or other virus) isolates to all currently available antiretroviral drugs. PHENOSENSE™ can obtain results with only one round of viral replication, thereby avoiding selection of subpopulations of virus that can occur during preparation of viral stocks required for assays that rely on fully infectious virus. Further, the results are both quantitative, measuring varying degrees of replication capacity, and sensitive, as the test can be performed on blood specimens with a viral load of about 500 copies/mL or above and can detect minority populations of some drug-resistant virus at concentrations of 10% or less of total viral population. Finally, the replication capacity results are reproducible and can vary by less than about 0.25 logs in about 95% of the assays performed.

PHENOSENSE™ can be used with nucleic acids from amplified viral gene sequences. As discussed below, the nucleic acid can be amplified from any sample known by one of skill in the art to contain a viral gene sequence, without limitation. For example, the sample can be a sample from a human or an animal infected with the virus or a sample from a culture of viral cells. In certain embodiments, the viral sample comprises a genetically modified laboratory strain. In other embodiments, the viral sample comprises a wild-type isolate.

A resistance test vector (“RTV”) can then be constructed by incorporating the amplified viral gene sequences into a replication defective viral vector by using any method known in the art of incorporating gene sequences into a vector. In one embodiment, restrictions enzymes and conventional cloning methods are used. See Sambrook et al., 2001, Molecular Cloning: A Laboratory Manual, Cold Spring Harbor Laboratory, 3^{rd} ed., NY; and Ausubel et al., 1989, Current Protocols in Molecular Biology, Greene Publishing Associates and Wiley Interscience, NY. In a preferred embodiment, ApaI and PinAI restriction enzymes are used. Preferably, the replication defective viral vector is the indicator gene viral vector (“IGVV”). In a preferred embodiment, the viral vector contains a means for detecting replication of the RTV. Preferably, the viral vector contains a luciferase expression cassette.

The assay can be performed by first co-transfecting host cells with RTV DNA and a plasmid that expresses the envelope proteins of another retrovirus, for example, amphotropic murine leukemia virus (MLV). Following transfection, viral particles can be harvested from the cell culture and used to infect fresh target cells in the presence of varying amounts of anti-viral drug(s). The completion of a single round of viral replication in the fresh target cells can be detected by the means for detecting replication contained in the vector. In a preferred embodiment, the completion of a single round of viral replication results in the production of luciferase. By monitoring the amount of, e.g., luciferase activity in the presence of the varying amounts of antiviral drug(s), a resistance curve can be constructed by plotting luciferase activity versus drug concentration. The susceptibility of an HIV, or population of HIV, can be determined by measuring the concentration of antiviral drug at which the luciferase activity detected is half-maximal. This number, the IC₅₀, provides a standard and convenient measure of drug resistance.

In preferred embodiments, PHENOSENSE™ is used to evaluate the d4T resistance or susceptibility phenotype of HV-1. In other embodiments, PHENOSENSE™ is used to evaluate the d4T resistance or susceptibility phenotype of HIV-2. In certain embodiments, the HIV-1 strain that is evaluated is a wild-type isolate of HIV-1. In other embodiments, the HIV-1 strain that is evaluated is a mutant strain of HIV-1. In certain embodiments, such mutant strains can be isolated from patients. In other embodiments, the mutant strains can be constructed by site-directed mutagenesis or other equivalent techniques known to one of skill in the art. In still other embodiments, the mutant strains can be isolated from cell culture. The cultures can comprise multiple passages through cell culture in the presence of antiviral compounds to select for mutations that accumulate in culture in the presence of such compounds.

In one embodiment, viral nucleic acid, for example, HIV-1 RNA is extracted from plasma samples, and a fragment of, or entire viral genes can be amplified by methods such as, but not limited to PCR. See, e.g., Hertogs et al., 1998, Antimicrob Agents Chemother 42(2):269-76. In one example, a 2.2-kb fragment containing the entire HIV-1 PR- and RT-coding sequence is amplified by nested reverse transcription-PCR. The pool of amplified nucleic acid, for example, the PR-RT-coding sequences, is then cotransfected into a host cell such as CD4+ T lymphocytes (MT4) with the pGEMT3deltaPRT plasmid from which most of the PR (codons 10 to 99) and RT (codons 1 to 482) sequences are deleted. Homologous recombination leads to the generation of chimeric viruses containing viral coding sequences, such as the PR- and RT-coding sequences derived from HIV-1 RNA in plasma. The replication capacities of the chimeric viruses can be determined by any cell viability assay known in the art, and compared to replication capacities of a statistically significant number of individual viral isolates to assess whether a virus has. For example, an MT4 cell-3-(4,5-dimethylthiazol-2-yl) -2,5-diphenyltetrazolium bromide-based cell viability assay can be used in an automated system that allows high sample throughput.

In another embodiment, competition assays can be used to assess replication capacity of one viral strain relative to another viral strain. For example, two infectious viral strains can be co-cultivated together in the same culture medium. See, e.g., Lu et al., 2001, JAIDS 27:7-13, which is incorporated by reference in its entirety. By monitoring the course of each viral strain's growth, the fitness of one strain relative to the other can be determined. By measuring many viruses' fitness relative to a single reference virus, an objective measure of each strain's fitness can be determined. These measurements of replication capacity can then be used according to the methods of the invention to identify targets for antiviral therapy.

Other assays for evaluating the phenotypic susceptibility of a virus to anti-viral drugs known to one of skill in the art can be adapted to determine replication capacity. See, e.g., Shi and Mellors, 1997, Antimicrob Agents Chemother. 41(12):2781-85; Gervaix et al., 1997, Proc Natl Acad Sci U.S.A. 94(9):4653-8; Race et al., 1999, AIDS 13:2061-2068, incorporated herein by reference in their entireties.

### 5.4.1. Detecting the Presence or Absence of Mutations in a Virus

The presence or absence of an mutation associated with d4T resistance or susceptibility according to the present invention in a virus can be determined by any means known in the art for detecting a mutation. The mutation can be detected in the viral gene that encodes a particular protein, or in the protein itself, i.e., in the amino acid sequence of the protein.

In one embodiment, the mutation is in the viral genome. Such a mutation can be in, for example, a gene encoding a viral protein, in a genetic element such as a cis or trans acting regulatory sequence of a gene encoding a viral protein, an intergenic sequence, or an intron sequence. The mutation can affect any aspect of the structure, function, replication or environment of the virus that changes its susceptibility to an anti-viral treatment and/or its replication capacity. In one embodiment, the mutation is in a gene encoding a viral protein that is the target of an currently available anti-viral treatment. In other embodiments, the mutation is in a gene or other genetic element that is not the target of a currently-available anti-viral treatment.

A mutation within a viral gene can be detected by utilizing any suitable technique known to one of skill in the art without limitation. Viral DNA or RNA can be used as the starting point for such assay techniques, and may be isolated according to standard procedures which are well known to those of skill in the art.

The detection of a mutation in specific nucleic acid sequences, such as in a particular region of a viral gene, can be accomplished by a variety of methods including, but not limited to, restriction-fragment-length-polymorphism detection based on allele-specific restriction-endonuclease cleavage (Kan and Dozy, 1978, Lancet ii:910-912), mismatch-repair detection (Faham and Cox, 1995, Genome Res 5:474-482), binding of MutS protein (Wagner et al., 1995, Nucl Acids Res 23:3944-3948), denaturing-gradient gel electrophoresis (Fisher et al., 1983, Proc. Natl. Acad. Sci. USA. 80:1579-83), single-strand-conformation-polymorphism detection (Orita et al., 1983, Genomics 5:874-879), RNAase cleavage at mismatched base-pairs (Myers et al., 1985, Science 230:1242), chemical (Cotton et al., 1988, Proc. Natl. Acad. Sci. U.S.A. 85:4397-4401) or enzymatic (Youil et al., 1995, Proc. Natl. Acad. Sci. U.S.A. 92:87-91) cleavage of heteroduplex DNA, methods based on oligonucleotide-specific primer extension (Syvänen et al., 1990, Genomics 8:684-692), genetic bit analysis (Nikiforov et al., 1994, Nucl Acids Res 22:4167-4175), oligonucleotide-ligation assay (Landegren et al., 1988, Science 241:1077), oligonucleotide-specific ligation chain reaction (“LCR”) (Barrany, 1991, Proc. Natl. Acad. Sci. U.S.A. 88:189-193), gap-LCR (Abravaya et al., 1995, Nucl Acids Res 23:675-682), radioactive or fluorescent DNA sequencing using standard procedures well known in the art, and peptide nucleic acid (PNA) assays (Orum et al., 1993, Nucl. Acids Res. 21:5332-5356; Thiede et al., 1996, Nucl. Acids Res. 24:983-984).

In addition, viral DNA or RNA may be used in hybridization or amplification assays to detect abnormalities involving gene structure, including point mutations, insertions, deletions and genomic rearrangements. Such assays may include, but are not limited to, Southern analyses (Southern, 1975, J. Mol. Biol. 98:503-517), single stranded conformational polymorphism analyses (SSCP) (Orita et al., 1989, Proc. Natl. Acad. Sci. USA 86:2766-2770), and PCR analyses (U.S. Pat. Nos. 4,683,202; 4,683,195; 4,800,159; and 4,965,188; PCR Strategies, 1995 Innis et al. (eds.), Academic Press, Inc.).

Such diagnostic methods for the detection of a gene-specific mutation can involve for example, contacting and incubating the viral nucleic acids with one or more labeled nucleic acid reagents including recombinant DNA molecules, cloned genes or degenerate variants thereof, under conditions favorable for the specific annealing of these reagents to their complementary sequences. Preferably, the lengths of these nucleic acid reagents are at least 15 to 30 nucleotides. After incubation, all non-annealed nucleic acids are removed from the nucleic acid molecule hybrid. The presence of nucleic acids which have hybridized, if any such molecules exist, is then detected. Using such a detection scheme, the nucleic acid from the virus can be immobilized, for example, to a solid support such as a membrane, or a plastic surface such as that on a microtiter plate or polystyrene beads. In this case, after incubation, non-annealed, labeled nucleic acid reagents of the type described above are easily removed. Detection of the remaining, annealed, labeled nucleic acid reagents is accomplished using standard techniques well-known to those in the art. The gene sequences to which the nucleic acid reagents have annealed can be compared to the annealing pattern expected from a normal gene sequence in order to determine whether a gene mutation is present.

These techniques can easily be adapted to provide high-throughput methods for detecting mutations in viral genomes. For example, a gene array from Affymetrix (Affymetrix, Inc., Sunnyvale, Calif.) can be used to rapidly identify genotypes of a large number of individual viruses. Affymetrix gene arrays, and methods of making and using such arrays, are described in, for example, U.S. Pat. Nos. 6,551,784, 6,548,257, 6,505,125, 6,489,114, 6,451,536, 6,410,229, 6,391,550, 6,379,895, 6,355,432, 6,342,355, 6,333,155, 6,308,170, 6,291,183, 6,287,850, 6,261,776, 6,225,625, 6,197,506, 6,168,948, 6,156,501, 6,141,096, 6,040,138, 6,022,963, 5,919,523, 5,837,832, 5,744,305, 5,834,758, and 5,631,734, each of which is hereby incorporated by reference in its entirety.

In addition, Ausubel et al., eds., Current Protocols in Molecular Biology, 2002, Vol. 4, Unit 25B, Ch. 22, which is hereby incorporated by reference in its entirety, provides further guidance on construction and use of a gene array for determining the genotypes of a large number of viral isolates. Finally, U.S. Pat. Nos. 6,670,124; 6,617,112; 6,309,823; 6,284,465; and 5,723,320, each of which is incorporated by reference in its entirety, describe related array technologies that can readily be adapted for rapid identification of a large number of viral genotypes by one of skill in the art.

Alternative diagnostic methods for the detection of gene specific nucleic acid molecules may involve their amplification, e.g., by PCR (U.S. Pat. Nos. 4,683,202; 4,683,195; 4,800,159; and 4,965,188; PCR Strategies, 1995 Innis et al. (eds.), Academic Press, Inc.), followed by the detection of the amplified molecules using techniques well known to those of skill in the art. The resulting amplified sequences can be compared to those which would be expected if the nucleic acid being amplified contained only normal copies of the respective gene in order to determine whether a gene mutation exists.

Additionally, the nucleic acid can be sequenced by any sequencing method known in the art. For example, the viral DNA can be sequenced by the dideoxy method of Sanger et al., 1977, Proc. Natl. Acad. Sci. USA 74:5463, as further described by Messing et al., 1981, Nuc. Acids Res. 9:309, or by the method of Maxam et al., 1980, Methods in Enzymology 65:499. See also the techniques described in Sambrook et al., 2001, Molecular Cloning: A Laboratory Manual, Cold Spring Harbor Laboratory, 3^{rd} ed., NY; and Ausubel et al., 1989, Current Protocols in Molecular Biology, Greene Publishing Associates and Wiley Interscience, NY.

Antibodies directed against the viral gene products, i.e., viral proteins or viral peptide fragments can also be used to detect mutations in the viral proteins. Alternatively, the viral protein or peptide fragments of interest can be sequenced by any sequencing method known in the art in order to yield the amino acid sequence of the protein of interest. An example of such a method is the Edman degradation method which can be used to sequence small proteins or polypeptides. Larger proteins can be initially cleaved by chemical or enzymatic reagents known in the art, for example, cyanogen bromide, hydroxylamine, trypsin or chymotrypsin, and then sequenced by the Edman degradation method.

### 5.4.2. Correlating Mutations with their Effects on Resistance or Susceptibility to d4T

Any method known in the art can be used to determine whether a mutation is correlated with d4T resistance or susceptibility. In one embodiment, univariate analysis is used to identify mutations correlated with altered replication capacity. Univariate analysis yields P values that indicate the statistical significance of the correlation. In such embodiments, the smaller the P value, the more significant the measurement. Preferably the P values will be less than 0.05. More preferably, P values will be less than 0.01. Even more preferably, the P value will be less than 0.005. P values can be calculated by any means known to one of skill in the art. In one embodiment, P values are calculated using Fisher's Exact Test. In another embodiment, P values can be calculated with Student's t-test. See, e.g., David Freedman, Robert Pisani & Roger Purves, 1980, STATISTICS, W. W. Norton, N.Y. In certain embodiments, P values can be calculated with both Fisher's Exact Test and Student's t-test. In such embodiments, P values calculated with both tests are preferably less than 0.05. However, a correlation with a P value that is less than 0.10 in one test but less than 0.05 in another test can still be considered to be a marginally significant correlation. Such mutations are suitable for further analysis with, for example, multivariate analysis. Alternatively, further univariate analysis can be performed on a larger sample set to confirm the significance of the correlation.

Further, an odds ratio can be calculated to determine whether a mutation associated with altered susceptibility to d4T correlates with resistance or susceptibility. Generally, calculation of odds rations depends on dividing the percentage of virus that comprise a particular mutation or mutations that are identified as resistant to d4T by the percentage of virus with the same mutation or mutations that are identified as susceptible to d4T. In certain embodiments, an odds ratio that is greater than one indicates that the mutation correlates with resistance to d4T. In certain embodiments, an odds ratio that is less than one indicates that the mutation correlates with susceptibility to d4T.

In yet another embodiment, multivariate analysis can be used to determine whether a mutation correlates with altered replication capacity. Any multivariate analysis known by one of skill in the art to be useful in calculating such a correlation can be used, without limitation. In certain embodiments, a statistically significant number of virus's resistance or susceptibility phenotypes, e.g., IC₅₀, can be determined. These IC₅₀ values can then be divided into groups that correspond to percentiles of the set of IC₅₀ values observed. For example, and not by way of limitation, the IC₅₀ values can be divided up into 21 groups. Each group corresponds to about 4.75% of the IC₅₀ values observed.

After assigning each virus's IC₅₀ value to the appropriate group, the genotype of that virus can be assigned to that group. By performing this method for all viral isolates, the number of instances of a particular mutation in a given percentile of replication capacity can be observed. This allows the skilled practitioner to identify mutations that correlate with altered replication capacity.

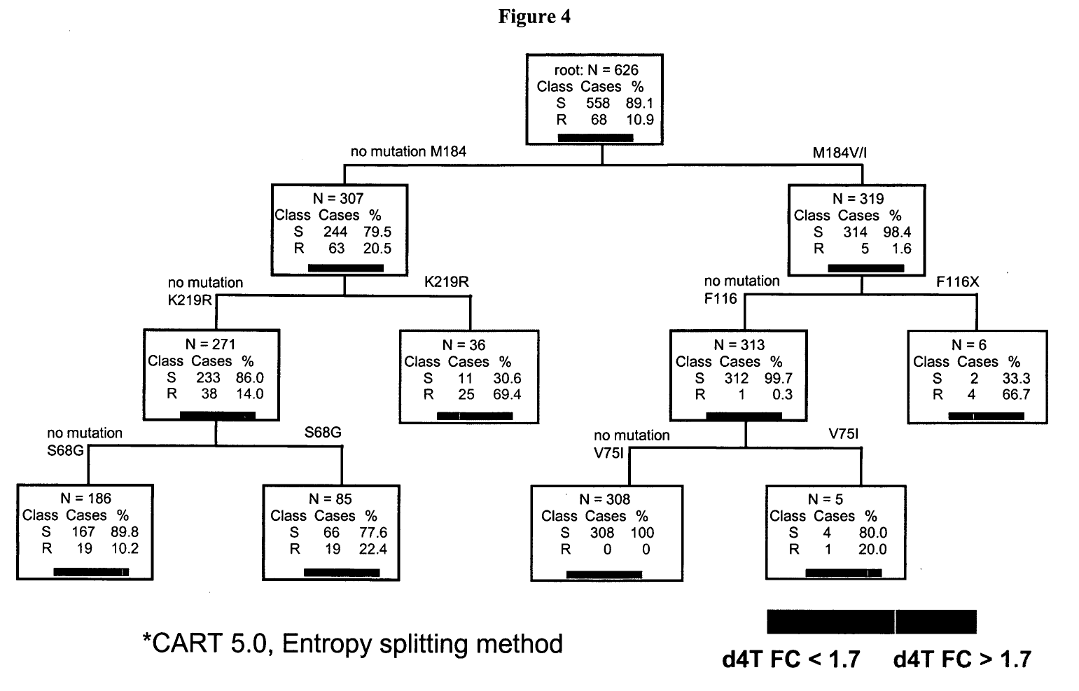

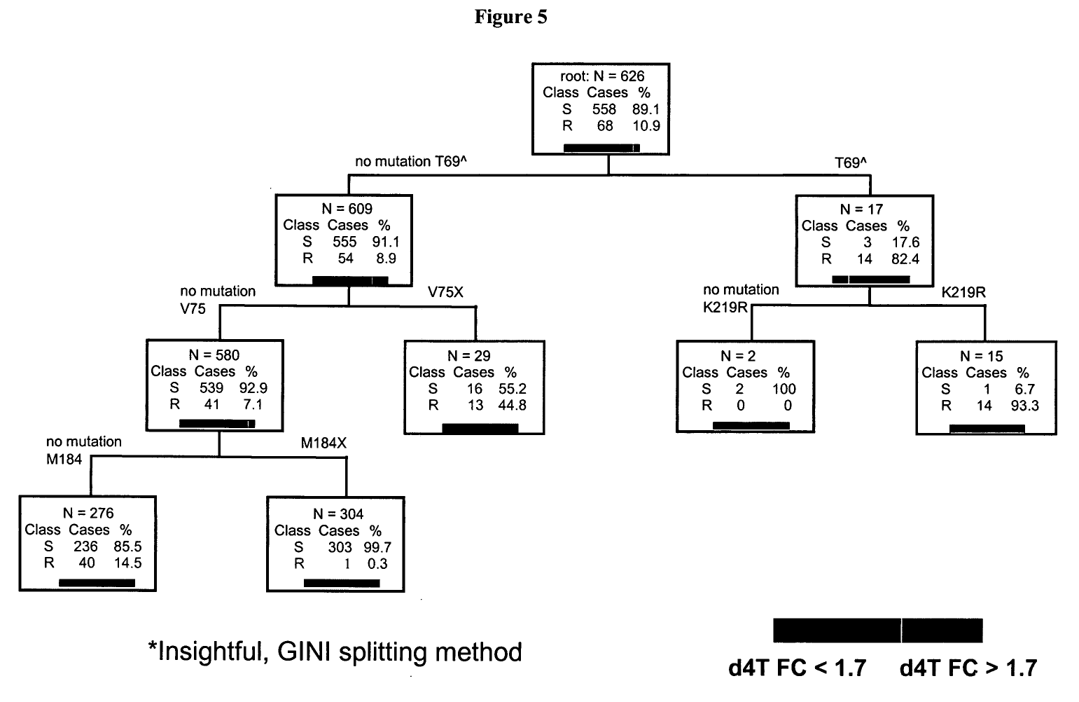

Finally, in yet another embodiment, regression analysis can be performed to identify mutations that best predict altered susceptibility to d4T. In such embodiments, regression analysis is performed on a statistically significant number of viral isolates for which genotypes and d4T susceptibility phenotypes have been determined. The analysis then identifies which mutations appear to best predict, e.g., most strongly correlate with, altered d4T susceptibility. Such analysis can then be used to construct rules for predicting replication capacity based upon knowledge of the genotype of a particular virus, described below. In certain embodiments, software such as, for example, CART 5.0 or Insightful Miner 3.0 can be used to perform the analysis that identifies the mutations that appear to best predict altered d4T susceptibility. Examples of such analyses are provided in FIGS. 4 and 5.

### 5.4.3. Computer-Implemented Methods for Determining Resistance or Susceptibility to d4T, and Articles Related Thereto

In another aspect, the present invention provides computer-implemented methods for identifying a target for antiviral therapy. In such embodiments, the methods of the invention are adapted to take advantage of the processing power of modem computers. One of skill in the art can readily adapt the methods in such a manner.

Therefore, in certain embodiments, the invention provides a computer-implemented method for determining that an HIV-1 is resistant to d4T, comprising inputting genetic information into a memory system of a computer, wherein the genetic information indicates that the HIV-1 has a gene encoding reverse transcriptase with a mutation at codon 65 in combination with a mutation at codon 40, 67, 69, 70, 75, 77, 116, 139, 169, 181, 190, 210, 215, 218, 219, 221, 224, or 227 of reverse transcriptase of the HIV-1, inputting a correlation between the presence of the mutations and resistance to d4T into the memory system of the computer, and determining that the HIV-1 is resistant to d4T.

In certain embodiments, the methods further comprise displaying that the HIV-1 is resistant to d4T on a display of the computer. In certain embodiments, the methods further comprise printing that the HIV-1 is resistant to d4T.

In another aspect, the invention provides a print-out indicating that an HIV-1 is resistant to d4T produced according to a method of the invention. In still another aspect, the invention provides a computer-readable medium comprising data indicating that an HIV-1 is resistant to d4T produced according to a method of the invention.

In other embodiments, the invention provides a computer-implemented method for determining that an HIV-1 is susceptible to d4T, comprising inputting genetic information into a memory system of a computer, wherein the genetic information indicates that the HIV-1 has a gene encoding reverse transcriptase with a mutation at codon 65 in combination with a mutation at codon 135 or 184 of reverse transcriptase of the HIV-1, inputting a correlation between the presence of the mutations and resistance to d4T into the memory system of the computer, and determining that the HIV-1 is susceptible to d4T.

In certain embodiments, the methods further comprise displaying that the HIV-1 is susceptible to d4T on a display of the computer. In certain embodiments, the methods further comprise printing that the HIV-1 is susceptible to d4T.

In another aspect, the invention provides a print-out indicating that an HIV-1 is susceptible to d4T produced according to a method of the invention.

In yet another aspect, the invention provides a computer-readable medium that comprises data indicating that an HIV-1 is susceptible to d4T produced according a method of the invention.

In still another aspect, the invention provides an article of manufacture that comprises computer-readable instructions for performing a method of the invention.

In yet another aspect, the invention provides a computer system that is configured to perform a method of the invention.

### 5.4.4. Viruses and Viral Samples

A mutation that affects d4T resistance or susceptibility according to the present invention can be present in any type of virus. For example, such mutations may be identified in any virus that infects animals known to one of skill in the art without limitation. In one embodiment of the invention, the virus includes viruses known to infect mammals, including dogs, cats, horses, sheep, cows etc. In certain embodiment, the virus is known to infect primates. In preferred embodiments, the virus is known to infect humans. Examples of such viruses that infect humans include, but are not limited to, human immunodeficiency virus (“HIV”), herpes simplex virus, cytomegalovirus virus, varicella zoster virus, other human herpes viruses, influenza A, B and C virus, respiratory syncytial virus, hepatitis A, B and C viruses, rhinovirus, and human papilloma virus. In certain embodiments, the virus is HCV. In other embodiments, the virus is HBV. In a preferred embodiment of the invention, the virus is HIV. Even more preferably, the virus is human immunodeficiency virus type 1 (“HIV-1”). The foregoing are representative of certain viruses for which there is presently available anti-viral chemotherapy and represent the viral families retroviridae, herpesviridae, orthomyxoviridae, paramxyxoviridae, picomaviridae, flaviviridae, pneumoviridae and hepadnaviridae. This invention can be used with other viral infections due to other viruses within these families as well as viral infections arising from viruses in other viral families for which there is or there is not a currently available therapy.

A mutation that affects d4T resistance or susceptibility according to the present invention can be found in a viral sample obtained by any means known in the art for obtaining viral samples. Such methods include, but are not limited to, obtaining a viral sample from a human or an animal infected with the virus or obtaining a viral sample from a viral culture. In one embodiment, the viral sample is obtained from a human individual infected with the virus. The viral sample could be obtained from any part of the infected individual's body or any secretion expected to contain the virus. Examples of such parts include, but are not limited to blood, serum, plasma, sputum, lymphatic fluid, semen, vaginal mucus and samples of other bodily fluids. In a preferred embodiment, the sample is a blood, serum or plasma sample.

In another embodiment, a mutation that affects d4T resistance or susceptibility according to the present invention is present in a virus that can be obtained from a culture. In some embodiments, the culture can be obtained from a laboratory. In other embodiments, the culture can be obtained from a collection, for example, the American Type Culture Collection.

In certain embodiments, a mutation that affects d4T resistance or susceptibility according to the present invention is present in a derivative of a virus. In one embodiment, the derivative of the virus is not itself pathogenic. In another embodiment, the derivative of the virus is a plasmid-based system, wherein replication of the plasmid or of a cell transfected with the plasmid is affected by the presence or absence of the selective pressure, such that mutations are selected that increase resistance to the selective pressure. In some embodiments, the derivative of the virus comprises the nucleic acids or proteins of interest, for example, those nucleic acids or proteins to be targeted by an anti-viral treatment. In one embodiment, the genes of interest can be incorporated into a vector. See, e.g., U.S. Pat. Nos. 5,837,464 and 6,242,187 and PCT publication, WO 99/67427, each of which is incorporated herein by reference. In certain embodiments, the genes can be those that encode for a protease or reverse transcriptase.

In another embodiment, the intact virus need not be used. Instead, a part of the virus incorporated into a vector can be used. Preferably that part of the virus is used that is targeted by an anti-viral drug.

In another embodiment, a mutation that affects d4T resistance or susceptibility according to the present invention is present in a genetically modified virus. The virus can be genetically modified using any method known in the art for genetically modifying a virus. For example, the virus can be grown for a desired number of generations in a laboratory culture. In one embodiment, no selective pressure is applied (i.e., the virus is not subjected to a treatment that favors the replication of viruses with certain characteristics), and new mutations accumulate through random genetic drift. In another embodiment, a selective pressure is applied to the virus as it is grown in culture (i.e., the virus is grown under conditions that favor the replication of viruses having one or more characteristics). In one embodiment, the selective pressure is an anti-viral treatment. Any known anti-viral treatment can be used as the selective pressure.

In certain embodiments, the virus is HIV and the selective pressure is a NNRTI. In another embodiment, the virus is HIV-1 and the selective pressure is a NNRTI. Any NNRTI can be used to apply the selective pressure. Examples of NNRTIs include, but are not limited to, nevirapine, delavirdine and efavirenz. By treating HIV cultured in vitro with a NNRTI, one can select for mutant strains of HIV that have an increased resistance to the NNRTI. The stringency of the selective pressure can be manipulated to increase or decrease the survival of viruses not having the selected-for characteristic.

In other embodiments, the virus is HIV and the selective pressure is a NRTI. In another embodiment, the virus is HIV-1 and the selective pressure is a NRTI. Any NRTI can be used to apply the selective pressure. Examples of NRTIs include, but are not limited to, AZT, ddI, ddC, d4T, 3TC, and abacavir. By treating HIV cultured in vitro with a NRTI, one can select for mutant strains of HIV that have an increased resistance to the NRTI. The stringency of the selective pressure can be manipulated to increase or decrease the survival of viruses not having the selected-for characteristic.

In still other embodiments, the virus is HIV and the selective pressure is a PI. In another embodiment, the virus is HIV-1 and the selective pressure is a PI. Any PI can be used to apply the selective pressure. Examples of PIs include, but are not limited to, saquinavir, ritonavir, indinavir, nelfinavir, amprenavir, lopinavir and atazanavir. By treating HIV cultured in vitro with a PI, one can select for mutant strains of HIV that have an increased resistance to the PI. The stringency of the selective pressure can be manipulated to increase or decrease the survival of viruses not having the selected-for characteristic.

In still other embodiments, the virus is HIV and the selective pressure is an entry inhibitor. In another embodiment, the virus is HIV-1 and the selective pressure is an entry inhibitor. Any entry inhibitor can be used to apply the selective pressure. An example of a entry inhibitor includes, but is not limited to, fusion inhibitors such as, for example, enfuvirtide. Other entry inhibitors include co-receptor inhibitors, such as, for example, AMD3100 (Anormed). Such co-receptor inhibitors can include any compound that interferes with an interaction between HIV and a co-receptor, e.g., CCR5 or CRCX4, without limitation. By treating HIV cultured in vitro with an entry inhibitor, one can select for mutant strains of HIV that have an increased resistance to the entry inhibitor. The stringency of the selective pressure can be manipulated to increase or decrease the survival of viruses not having the selected-for characteristic.

In another aspect, a mutation that affects d4T resistance or susceptibility according to the present invention is made by mutagenizing a virus, a viral genome, or a part of a viral genome. Any method of mutagenesis known in the art can be used for this purpose. In certain embodiments, the mutagenesis is essentially random. In certain embodiments, the essentially random mutagenesis is performed by exposing the virus, viral genome or part of the viral genome to a mutagenic treatment. In another embodiment, a gene that encodes a viral protein that is the target of an anti-viral therapy is mutagenized. Examples of essentially random mutagenic treatments include, for example, exposure to mutagenic substances (e.g., ethidium bromide, ethylmethanesulphonate, ethyl nitroso urea (ENU) etc.) radiation (e.g., ultraviolet light), the insertion and/or removal of transposable elements (e.g., Tn5, Tn10), or replication in a cell, cell extract, or in vitro replication system that has an increased rate of mutagenesis. See, e.g., Russell et al., 1979, Proc. Nat. Acad. Sci. USA 76:5918-5922; Russell, W., 1982, Environmental Mutagens and Carcinogens: Proceedings of the Third International Conference on Environmental Mutagens. One of skill in the art will appreciate that while each of these methods of mutagenesis is essentially random, at a molecular level, each has its own preferred targets.

In another aspect, a mutation that affects d4T resistance or susceptibility is made using site-directed mutagenesis. Any method of site-directed mutagenesis known in the art can be used (see e.g., Sambrook et al., 2001, Molecular Cloning: A Laboratory Manual, Cold Spring Harbor Laboratory, 3^{rd} ed., NY; and Ausubel et al., 1989, Current Protocols in Molecular Biology, Greene Publishing Associates and Wiley Interscience, N.Y.). See, e.g., Sarkar and Sommer, 1990, Biotechniques, 8:404-407. The site directed mutagenesis can be directed to, e.g., a particular gene or genomic region, a particular part of a gene or genomic region, or one or a few particular nucleotides within a gene or genomic region. In one embodiment, the site directed mutagenesis is directed to a viral genomic region, gene, gene fragment, or nucleotide based on one or more criteria. In one embodiment, a gene or a portion of a gene is subjected to site-directed mutagenesis because it encodes a protein that is known or suspected to be a target of an anti-viral therapy, e.g., the gene encoding the HIV reverse transcriptase. In another embodiment, a portion of a gene, or one or a few nucleotides within a gene, are selected for site-directed mutagenesis. In one embodiment, the nucleotides to be mutagenized encode amino acid residues that are known or suspected to interact with an anti-viral compound. In another embodiment, the nucleotides to be mutagenized encode amino acid residues that are known or suspected to be mutated in viral strains having an altered replication capacity. In another embodiment, the mutagenized nucleotides encode amino acid residues that are adjacent to or near in the primary sequence of the protein residues known or suspected to interact with an anti-viral compound or known or suspected to be mutated in viral strains having an altered replication capacity. In another embodiment, the mutagenized nucleotides encode amino acid residues that are adjacent to or near to in the secondary, tertiary or quaternary structure of the protein residues known or suspected to interact with an anti-viral compound or known or suspected to be mutated in viral strains having an altered replication capacity. In another embodiment, the mutagenized nucleotides encode amino acid residues in or near the active site of a protein that is known or suspected to bind to an anti-viral compound.

### 6. EXAMPLES

### 6.1. Example 1

### Measuring d4T Resistance or Susceptibility Using Resistance Test Vectors

This example provides methods and compositions for accurately and reproducibly measuring the resistance or sensitivity of HIV-1 to antiretroviral drugs including, for example, NRTIs such as d4T. The methods for measuring resistance or susceptibility to such drugs can be adapted to other HIV strains, such as HIV-2, or to other viruses, including, but not limited to hepadnaviruses (e.g., human hepatitis B virus), flaviviruses (e.g., human hepatitis C virus) and herpesviruses (e.g., human cytomegalovirus).

Drug resistance tests can be carried out using the methods for phenotypic drug susceptibility and resistance tests described in U.S. Pat. No. 5,837,464 (International Publication Number WO 97/27319) which is hereby incorporated by reference in its entirety, or according to the protocol that follows.

Patient-derived segment(s) corresponding to the HIV protease and reverse transcriptase coding regions were amplified by the reverse transcription-polymerase chain reaction method (RT-PCR) using viral RNA isolated from viral particles present in the plasma or serum of HIV-infected individuals as follows. Viral RNA was isolated from the plasma or serum using oligo-dT magnetic beads (Dynal Biotech, Oslo, Norway), followed by washing and elution of viral RNA. The RT-PCR protocol was divided into two steps. A retroviral reverse transcriptase (e.g. Moloney MuLV reverse transcriptase (Roche Molecular Systems, Inc., Branchburg, N.J.; Invitrogen, Carlsbad, Calif.), or avian myeloblastosis virus (AMV) reverse transcriptase, (Boehringer Mannheim, Indianapolis, Ind.), or ) was used to copy viral RNA into cDNA. The cDNA was then amplified using a thermostable DNA polymerase (e.g. Taq (Roche Molecular Systems, Inc., Branchburg, N.J.), Tth (Roche Molecular Systems, Inc., Branchburg, N.J.), PRIMEZYME™ (isolated from Thermus brockianus, Biometra, Gottingen, Germany)) or a combination of thermostable polymerases as described for the performance of “long PCR” (Barnes, W. M., 1994, Proc. Natl. Acad. Sci, USA 91, 2216-20) (e.g. Expand High Fidelity PCR System (Taq+Pwo), (Boehringer Mannheim. Indianapolis, Ind.); GENEAMP XL™ PCR kit (Tth +Vent), (Roche Molecular Systems, Inc., Branchburg, N.J.); or ADVANTAGE II®, Clontech, Palo Alto, Calif.)

PCR primers were designed to introduce ApaI and PinA1 recognition sites into the 5′ or 3′ end of the PCR product, respectively.

Replication capacity test vectors incorporating the “test” patient-derived segments were constructed as described in U.S. Pat. No. 5,837,464 using an amplified DNA product of 1.5 kB prepared by RT-PCR using viral RNA as a template and oligonucleotides PDS Apa, PDS Age, PDS PCR6, Apa-gen, Apa-c, Apa-f, Age-gen, Age-a, RT-ad, RT-b, RT-c, RT-f, and/or RT-g as primers, followed by digestion with ApaI and AgeI or the isoschizomer PinA1. To ensure that the plasmid DNA corresponding to the resultant fitness test vector comprises a representative sample of the HIV viral quasi-species present in the serum of a given patient, many (>250) independent E. coli transformants obtained in the construction of a given fitness test vector are pooled and used for the preparation of plasmid DNA.

A packaging expression vector encoding an amphotrophic MuLV 4070A env gene product enables production in a replication capacity test vector host cell of replication capacity test vector viral particles which can efficiently infect human target cells. Replication capacity test vectors encoding all HIV genes with the exception of env were used to transfect a packaging host cell (once transfected the host cell is referred to as a fitness test vector host cell). The packaging expression vector which encodes the amphotrophic MuLV 4070A env gene product is used with the replication capacity test vector to enable production in the replication capacity test vector host cell of infectious pseudotyped replication capacity test vector viral particles.

Drug resistance tests performed with resistance test vectors were carried out using packaging host and target host cells consisting of the human embryonic kidney cell line 293.

Resistance tests were carried out with resistance test vectors using two host cell types. Resistance test vector viral particles were produced by a first host cell (the resistance test vector host cell) that was prepared by transfecting a packaging host cell with the resistance test vector and the packaging expression vector. The resistance test vector viral particles were then used to infect a second host cell (the target host cell) in which the expression of the indicator gene is measured.

The resistance test vectors containing a functional luciferase gene cassette were constructed as described above and host cells were transfected with the resistance test vector DNA. The resistance test vectors contained patient-derived reverse transcriptase and protease DNA sequences that encode proteins which were either susceptible or resistant to the antiretroviral agents, such as, for example, NRTIs, NNRTIs, and PIs.

The amount of luciferase activity detected in infected cells is used as a direct measure of “infectivity,” i.e., the ability of the virus to complete a single round of replication. Thus, drug resistance or sensitivity can be determined by plotting the amount of luciferase activity produced by patient derived viruses in the presence of varying concentrations of the antiviral drug. By identifying the concentration of drug at which luciferase activity is half-maximum, the IC₅₀ of the virus from which patient-derived segment(s) were obtained for the antiretroviral agent can be determined.

Host (293) cells were seeded in 10-cm-diameter dishes and were transfected one day after plating with resistance test vector plasmid DNA and the envelope expression vector. Transfections were performed using a calcium-phosphate co-precipitation procedure. The cell culture media containing the DNA precipitate was replaced with fresh medium, from one to 24 hours, after transfection. Cell culture medium containing resistance test vector viral particles was harvested one to four days after transfection and was passed through a 0.45-mm filter before optional storage at −80° C. Before infection, target cells (293 cells) were plated in cell culture media. Control infections were performed using cell culture media from mock transfections (no DNA) or transfections containing the resistance test vector plasmid DNA without the envelope expression plasmid. One to three or more days after infection the media was removed and cell lysis buffer (Promega Corp.; Madison, Wis.) was added to each well. Cell lysates were assayed for luciferase activity. Alternatively, cells were lysed and luciferase was measured by adding Steady-Glo (Promega Corp.; Madison, Wis.) reagent directly to each well without aspirating the culture media from the well. The amount of luciferase activity produced in infected cells is normalized to adjust for variation in transfection efficiency in the transfected host cells by measuring the luciferase activity in the transfected cells, which is not dependent on viral gene functions, and adjusting the luciferase activity from infected cell accordingly.

### 6.2. Example 2

### Identifying Mutations Correlated with Resistance or Susceptibility to d4T

This example provides methods and compositions for identifying mutations that correlate with resistance or susceptibility to d4T. Resistance test vectors were constructed and used as described in Example 1. Resistance test vectors derived from patient samples or clones derived from the resistance test vector pools were tested in a resistance assay to determine accurately and quantitatively the relative d4T resistance or susceptibility compared to the median observed resistance or susceptibility.

Genotypic Analysis of Patient HIV Samples:

Resistance test vector DNAs, either pools or clones, can be analyzed by any genotyping method, e.g., as described above. In this example, patient HIV sample sequences were determined using viral RNA purification, RT/PCR and ABI chain terminator automated sequencing. The sequence that was determined was compared to that of a reference sequence, NL4-3. The genotype was examined for sequences that were different from the reference or pre-treatment sequence and correlated to the observed IC₅₀ for d4T.

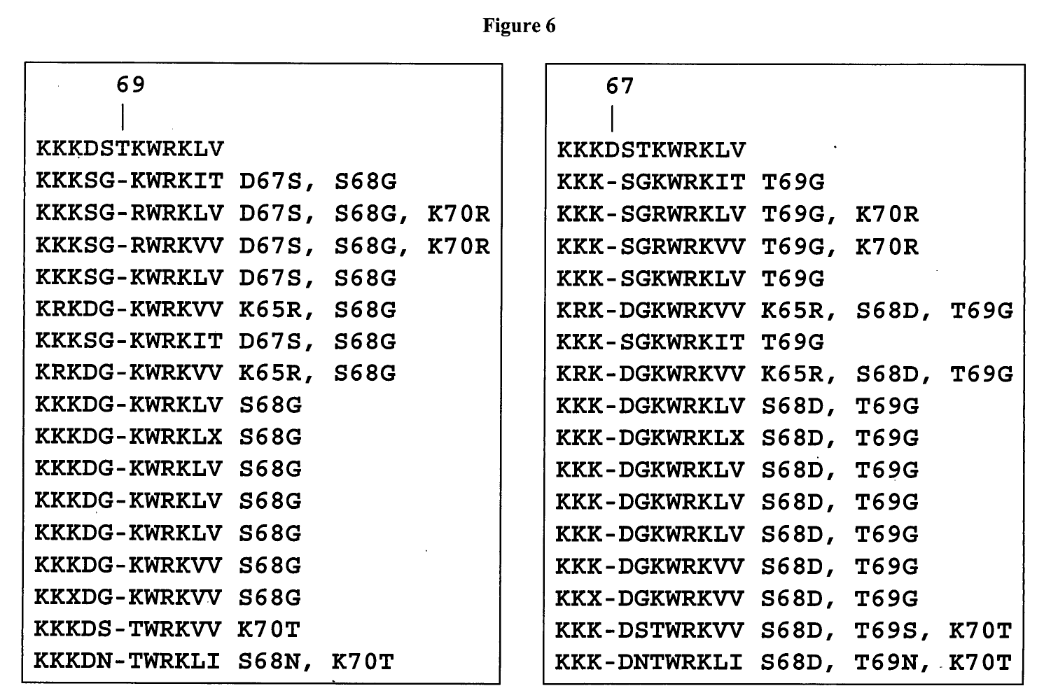

It should be noted that the T69 deletion can be assigned to a slightly different genotype. In particular, some reports identify this deletion as a deletion at D67. See, e.g., Imamichi et al., 200, J. Virol. 74:1023-1028 and Imamichi et al., 2001, J. Virol. 75:3988-3992. A genotype that contains D67A and T69G is exactly equivalent to D67S, S68G, and T69A. See FIG. 6.

Correlation of Resistance or Susceptibility to d4T and Mutations:

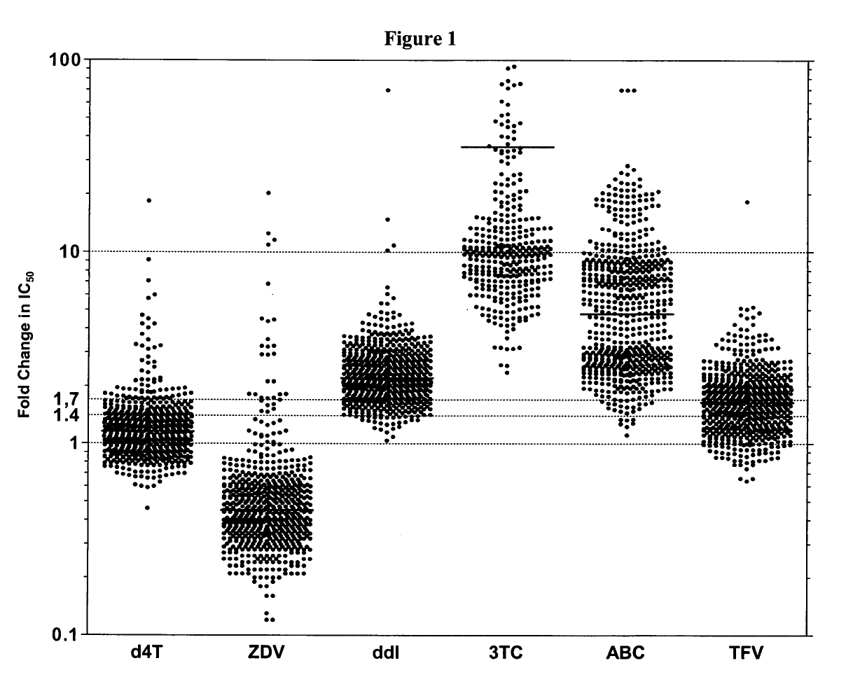

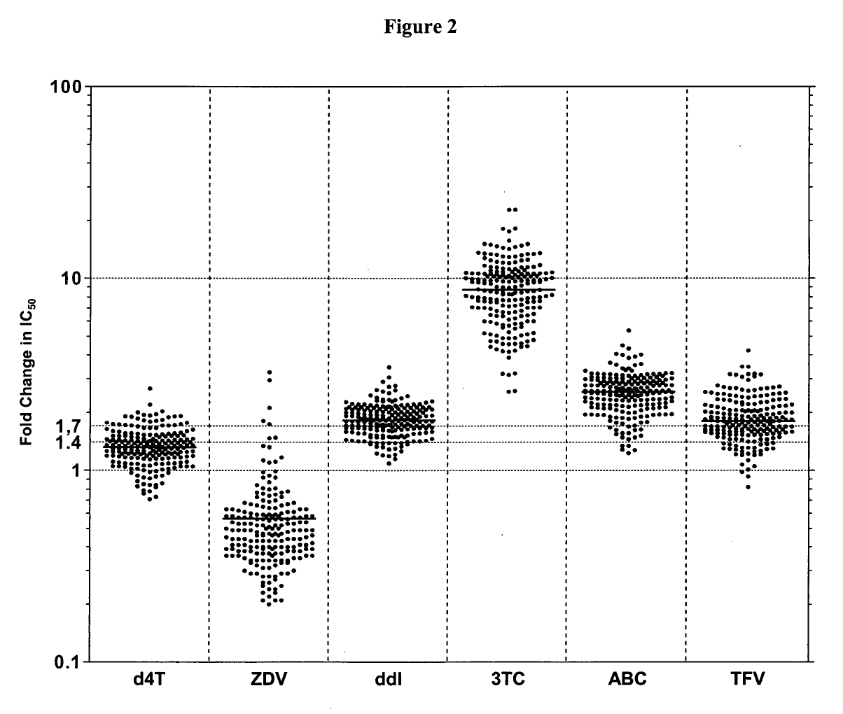

To identify mutations associated with d4T resistance or susceptibility in the presence of K65R, the following analysis was performed. A collection of 626 samples that comprise the K65R mutation but do not comprise the Q151M or T69ins mutations were identified and their d4T susceptibility phenotype was determined as described above. The distribution of IC₅₀ values observed for the 626 individual viruses for an array of NRTIs, including d4T, is presented as FIG. 1, while the distribution of IC₅₀ values for the NRTIs of observed for 202 viruses that comprise only the K65R mutation in RT is presented as FIG. 2. Viruses that exhibit an IC₅₀ 1.7 fold higher than wild-type virus were designated as resistant to d4T, while viruses with IC₅₀ values below this threshold were designated as susceptible. Most of the viruses comprising only the K65R mutation exhibit an IC₅₀ less than 1.7 fold higher than wild-type, while some exhibit an IC₅₀ greater than 1.7 fold higher than wild-type.

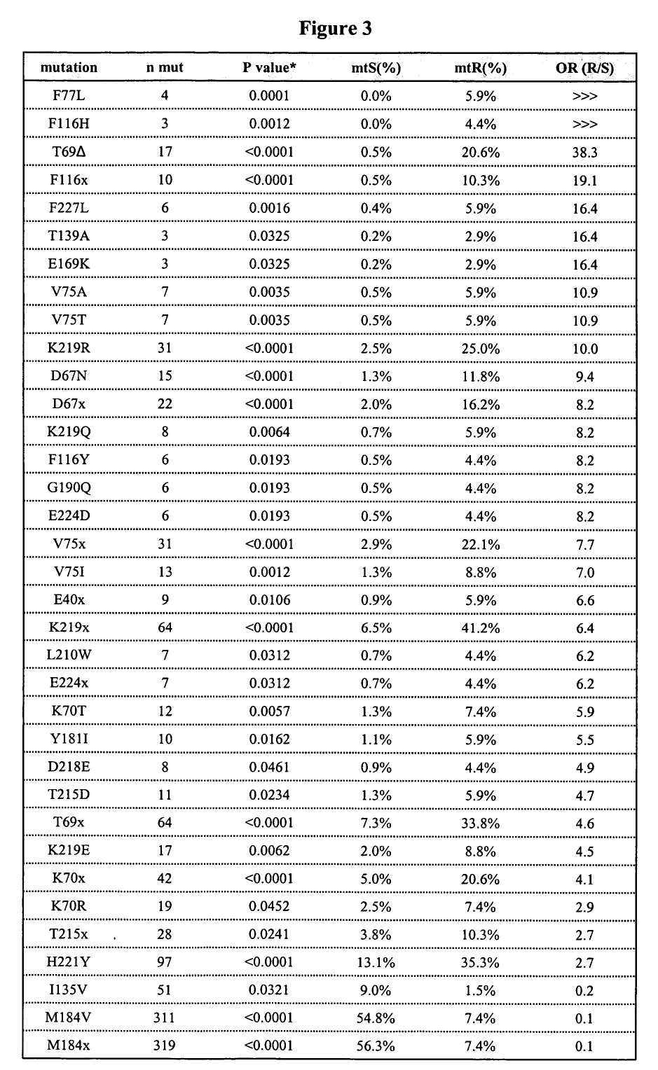

Next, Fisher's Exact test was used to identify mutations associated with resistance or susceptibility to d4T in the presence of the K65R mutation. As discussed above, viruses that exhibit an IC₅₀ 1.7 fold higher than wild-type virus were designated as resistant to d4T, while viruses with IC₅₀ values below this threshold were designated as susceptible. Significant correlations were defined to exhibit p-values less than 0.05 and odds ratios (determined by dividing the fraction of viruses with a particular mutation exhibiting resistance by the fraction of viruses with the mutation exhibiting susceptibility) greater than 2 or less than 0.5. Results from this analysis are summarized in FIG. 3.

In addition, the prediction power of the proportion of samples with a given mutation was tested in a regression tree using CART 5.0 software (Salford Systems, San Diego Calif.) or Insightful Miner 3.0 (Insightful Corp., Seattle Wash.). This procedure identifies the variable with the greatest ability to separate samples into two groups based on their d4T resistance or susceptibility. All 626 samples were examined in this analysis. For example, the presence of a mutation at codon 184 the best separator variable in the CART 5.0 analysis (See FIG. 4). However, the deletion at codon 69 was the best separator variable in the Insightful Miner 3.0 analysis (See FIG. 5).

Mutations Associated with d4T Resistance or Susceptibility

The experiments described above identified a number of mutations that significantly correlate with either resistance or susceptibility to d4T in the presence of K65R. The specific mutations identified as most significantly correlated with d4T resistance or susceptibility are presented in FIG. 3, together with statistical data showing the correlations between the mutations and altered replication capacity. The complete set of mutations detected together with the K65R mutation in the sample set is listed in Table 1, below, along with the p values and odds ratios associated with these mutations. All mutations that do not indicate an amino acid following the codon position (e.g., Q151) were mixtures of two or more mutations at the corresponding position, indicating that the patient from which the sample was obtained was infected with a mixed population of viruses as to that position.

TABLE 1

| | number | | | | |
|---|---|---|---|---|---|
| mutation | observed | P value | mtS(%) | mtR(%) | R/S |
| F77L | 4 | 0.0001 | 0.0% | 5.9% | 0.0 |
| F116 | 10 | 0.0001 | 0.5% | 10.3% | 19.1 |
| D67N | 15 | 0.0001 | 1.3% | 11.8% | 9.4 |
| T69\hat{ } | 17 | 0.0001 | 0.5% | 20.6% | 38.3 |
| D67 | 22 | 0.0001 | 2.0% | 16.2% | 8.2 |
| K219R | 31 | 0.0001 | 2.5% | 25.0% | 10.0 |
| V75 | 31 | 0.0001 | 2.9% | 22.1% | 7.7 |
| K70 | 42 | 0.0001 | 5.0% | 20.6% | 4.1 |
| K219 | 64 | 0.0001 | 6.5% | 41.2% | 6.4 |
| T69 | 64 | 0.0001 | 7.3% | 33.8% | 4.6 |
| H221Y | 97 | 0.0001 | 13.1% | 35.3% | 2.7 |
| S68G | 197 | 0.0001 | 28.9% | 52.9% | 1.8 |
| M184V | 311 | 0.0001 | 54.8% | 7.4% | 0.1 |
| M184 | 319 | 0.0001 | 56.3% | 7.4% | 0.1 |
| F116H | 3 | 0.0012 | 0.0% | 4.4% | >>> |
| V75I | 13 | 0.0012 | 1.3% | 8.8% | 7.0 |
| S68 | 288 | 0.0012 | 43.7% | 64.7% | 1.5 |
| F227L | 6 | 0.0016 | 0.4% | 5.9% | 16.4 |
| V75A | 7 | 0.0035 | 0.5% | 5.9% | 10.9 |
| V75T | 7 | 0.0035 | 0.5% | 5.9% | 10.9 |
| Y181 | 232 | 0.0051 | 35.1% | 52.9% | 1.5 |
| K70T | 12 | 0.0057 | 1.3% | 7.4% | 5.9 |
| K219E | 17 | 0.0062 | 2.0% | 8.8% | 4.5 |
| Q207 | 175 | 0.0062 | 26.2% | 42.6% | 1.6 |
| K219Q | 8 | 0.0064 | 0.7% | 5.9% | 8.2 |
| E40 | 9 | 0.0106 | 0.9% | 5.9% | 6.6 |
| Q151 | 2 | 0.0116 | 0.0% | 2.9% | >>> |
| K223Q | 2 | 0.0116 | 0.0% | 2.9% | >>> |
| F214 | 142 | 0.0134 | 21.1% | 35.3% | 1.7 |
| Y181I | 10 | 0.0162 | 1.1% | 5.9% | 5.5 |
| G190 | 137 | 0.0188 | 20.4% | 33.8% | 1.7 |
| F116Y | 6 | 0.0193 | 0.5% | 4.4% | 8.2 |
| G190Q | 6 | 0.0193 | 0.5% | 4.4% | 8.2 |
| E224D | 6 | 0.0193 | 0.5% | 4.4% | 8.2 |
| A62 | 86 | 0.0232 | 12.5% | 23.5% | 1.9 |
| T215D | 11 | 0.0234 | 1.3% | 5.9% | 4.7 |
| T215 | 28 | 0.0241 | 3.8% | 10.3% | 2.7 |
| L210W | 7 | 0.0312 | 0.7% | 4.4% | 6.2 |
| E224 | 7 | 0.0312 | 0.7% | 4.4% | 6.2 |
| Y181C | 220 | 0.0319 | 33.7% | 47.1% | 1.4 |
| I135V | 51 | 0.0321 | 9.0% | 1.5% | 0.2 |
| T139A | 3 | 0.0325 | 0.2% | 2.9% | 16.4 |
| E169K | 3 | 0.0325 | 0.2% | 2.9% | 16.4 |
| G190A | 86 | 0.0408 | 12.7% | 22.1% | 1.7 |
| F214L | 140 | 0.0446 | 21.1% | 32.4% | 1.5 |
| K70R | 19 | 0.0452 | 2.5% | 7.4% | 2.9 |
| D218E | 8 | 0.0461 | 0.9% | 4.4% | 4.9 |
| L74 | 38 | 0.0541 | 5.4% | 11.8% | 2.2 |
| L74I | 4 | 0.0604 | 0.4% | 2.9% | 8.2 |
| K223 | 4 | 0.0604 | 0.4% | 2.9% | 8.2 |
| I257L | 4 | 0.0604 | 0.4% | 2.9% | 8.2 |
| L301M | 4 | 0.0604 | 0.4% | 2.9% | 8.2 |
| V35 | 227 | 0.0610 | 34.9% | 47.1% | 1.3 |
| I135 | 260 | 0.0680 | 42.8% | 30.9% | 0.7 |
| V106 | 60 | 0.0766 | 8.8% | 16.2% | 1.8 |
| Q207E | 106 | 0.0847 | 15.9% | 25.0% | 1.6 |
| T296S | 16 | 0.0848 | 2.2% | 5.9% | 2.7 |
| A62V | 83 | 0.0854 | 12.4% | 20.6% | 1.7 |
| L74V | 35 | 0.0891 | 5.0% | 10.3% | 2.1 |
| L228R | 23 | 0.0929 | 3.2% | 7.4% | 2.3 |
| T27S | 5 | 0.0937 | 0.5% | 2.9% | 5.5 |
| T27 | 5 | 0.0937 | 0.5% | 2.9% | 5.5 |
| I257 | 5 | 0.0937 | 0.5% | 2.9% | 5.5 |
| E203 | 17 | 0.1021 | 2.3% | 5.9% | 2.5 |
| T296 | 17 | 0.1021 | 2.3% | 5.9% | 2.5 |
| K104R | 11 | 0.1071 | 1.4% | 4.4% | 3.1 |
| V106A | 11 | 0.1071 | 1.4% | 4.4% | 3.1 |
| Q151L | 1 | 0.1086 | 0.0% | 1.5% | 0.0 |
| Y188 | 42 | 0.1164 | 6.1% | 11.8% | 1.9 |
| A272P | 361 | 0.1192 | 58.8% | 48.5% | 0.8 |
| A98S | 59 | 0.1235 | 8.8% | 14.7% | 1.7 |
| Y188L | 30 | 0.1241 | 4.3% | 8.8% | 2.1 |
| K122 | 331 | 0.1247 | 51.8% | 61.8% | 1.2 |
| S251T | 6 | 0.1309 | 0.7% | 2.9% | 4.1 |
| V106M | 12 | 0.1320 | 1.6% | 4.4% | 2.7 |
| L283I | 65 | 0.1365 | 9.7% | 16.2% | 1.7 |
| L283 | 65 | 0.1365 | 9.7% | 16.2% | 1.7 |
| A272 | 392 | 0.1458 | 63.6% | 54.4% | 0.9 |
| T200 | 243 | 0.1485 | 37.8% | 47.1% | 1.2 |
| D250E | 69 | 0.1529 | 10.4% | 16.2% | 1.6 |
| Q207A | 13 | 0.1588 | 1.8% | 4.4% | 2.5 |
| T200A | 184 | 0.1609 | 28.5% | 36.8% | 1.3 |
| E138 | 24 | 0.1676 | 3.4% | 7.4% | 2.2 |
| T69D | 7 | 0.1708 | 0.9% | 2.9% | 3.3 |
| Y188H | 7 | 0.1708 | 0.9% | 2.9% | 3.3 |
| L228 | 38 | 0.1709 | 5.6% | 10.3% | 1.9 |
| Y115F | 113 | 0.1825 | 18.8% | 11.8% | 0.6 |
| Y115 | 113 | 0.1825 | 18.8% | 11.8% | 0.6 |
| G190T | 2 | 0.2056 | 0.2% | 1.5% | 8.2 |
| I195L | 2 | 0.2056 | 0.2% | 1.5% | 8.2 |
| T215E | 2 | 0.2056 | 0.2% | 1.5% | 8.2 |
| S68N | 134 | 0.2091 | 22.2% | 14.7% | 0.7 |
| V8I | 8 | 0.2123 | 1.1% | 2.9% | 2.7 |
| V8 | 8 | 0.2123 | 1.1% | 2.9% | 2.7 |
| K104 | 15 | 0.2166 | 2.2% | 4.4% | 2.1 |
| L210 | 15 | 0.2166 | 2.2% | 4.4% | 2.1 |
| R284K | 15 | 0.2166 | 2.2% | 4.4% | 2.1 |
| R284 | 15 | 0.2166 | 2.2% | 4.4% | 2.1 |
| P294Q | 15 | 0.2166 | 2.2% | 4.4% | 2.1 |
| A288S | 71 | 0.2211 | 10.8% | 16.2% | 1.5 |
| Q174 | 72 | 0.2249 | 10.9% | 16.2% | 1.5 |
| D250 | 72 | 0.2249 | 10.9% | 16.2% | 1.5 |
| E53 | 16 | 0.2399 | 2.9% | 0.0% | 0.0 |
| T139 | 9 | 0.2547 | 1.3% | 2.9% | 2.3 |
| E203K | 9 | 0.2547 | 1.3% | 2.9% | 2.3 |
| L228H | 9 | 0.2547 | 1.3% | 2.9% | 2.3 |
| K43 | 20 | 0.2590 | 2.9% | 5.9% | 2.1 |
| S48E | 3 | 0.2922 | 0.4% | 1.5% | 4.1 |
| T215Y | 3 | 0.2922 | 0.4% | 1.5% | 4.1 |
| K219H | 3 | 0.2922 | 0.4% | 1.5% | 4.1 |
| A98 | 67 | 0.2960 | 10.2% | 14.7% | 1.4 |
| W88C | 10 | 0.2974 | 1.4% | 2.9% | 2.1 |
| W88 | 10 | 0.2974 | 1.4% | 2.9% | 2.1 |
| L301 | 10 | 0.2974 | 1.4% | 2.9% | 2.1 |
| I202 | 68 | 0.3004 | 10.4% | 14.7% | 1.4 |
| M41L | 24 | 0.3178 | 3.6% | 5.9% | 1.6 |
| M41 | 25 | 0.3366 | 3.8% | 5.9% | 1.6 |
| V90I | 48 | 0.3426 | 7.3% | 10.3% | 1.4 |
| V90 | 48 | 0.3426 | 7.3% | 10.3% | 1.4 |
| V108 | 49 | 0.3440 | 8.2% | 4.4% | 0.5 |
| V118I | 27 | 0.3447 | 4.7% | 1.5% | 0.3 |
| V118 | 28 | 0.3475 | 4.8% | 1.5% | 0.3 |
| I178M | 86 | 0.3496 | 13.3% | 17.6% | 1.3 |
| K49R | 29 | 0.3523 | 5.0% | 1.5% | 0.3 |
| K49 | 29 | 0.3523 | 5.0% | 1.5% | 0.3 |
| V276T | 4 | 0.3694 | 0.5% | 1.5% | 2.7 |
| Q174K | 32 | 0.3777 | 4.8% | 7.4% | 1.5 |
| M184I | 13 | 0.3797 | 2.3% | 0.0% | 0.0 |
| E53D | 14 | 0.3835 | 2.5% | 0.0% | 0.0 |
| G190S | 33 | 0.3892 | 5.0% | 7.4% | 1.5 |
| V179D | 15 | 0.3911 | 2.7% | 0.0% | 0.0 |
| K173 | 65 | 0.4012 | 10.0% | 13.2% | 1.3 |
| T286 | 185 | 0.4023 | 29.0% | 33.8% | 1.2 |
| K281R | 69 | 0.4121 | 11.5% | 7.4% | 0.6 |
| K281 | 69 | 0.4121 | 11.5% | 7.4% | 0.6 |
| V292I | 73 | 0.4226 | 11.3% | 14.7% | 1.3 |
| T200I | 40 | 0.4261 | 6.1% | 8.8% | 1.4 |
| V292 | 74 | 0.4274 | 11.5% | 14.7% | 1.3 |
| K173E | 18 | 0.4320 | 2.7% | 4.4% | 1.6 |
| K103N | 250 | 0.4341 | 40.5% | 35.3% | 0.9 |
| K103 | 271 | 0.4371 | 43.9% | 38.2% | 0.9 |
| E36A | 5 | 0.4384 | 0.7% | 1.5% | 2.1 |
| V111I | 5 | 0.4384 | 0.7% | 1.5% | 2.1 |
| N175H | 5 | 0.4384 | 0.7% | 1.5% | 2.1 |
| E204K | 5 | 0.4384 | 0.7% | 1.5% | 2.1 |
| K219N | 5 | 0.4384 | 0.7% | 1.5% | 2.1 |
| P243S | 5 | 0.4384 | 0.7% | 1.5% | 2.1 |
| K32 | 19 | 0.4499 | 2.9% | 4.4% | 1.5 |
| T165I | 43 | 0.4507 | 6.6% | 8.8% | 1.3 |
| I178 | 150 | 0.4518 | 23.5% | 27.9% | 1.2 |
| A288 | 84 | 0.4543 | 13.1% | 16.2% | 1.2 |
| V179 | 86 | 0.4589 | 14.2% | 10.3% | 0.7 |
| K166 | 46 | 0.4609 | 7.7% | 4.4% | 0.6 |
| V108I | 48 | 0.4657 | 8.1% | 4.4% | 0.5 |
| Q174R | 20 | 0.4692 | 3.0% | 4.4% | 1.4 |
| Q278H | 20 | 0.4692 | 3.0% | 4.4% | 1.4 |
| T286A | 179 | 0.4788 | 28.1% | 32.4% | 1.1 |
| S251 | 21 | 0.4897 | 3.2% | 4.4% | 1.4 |
| K32R | 6 | 0.4999 | 0.9% | 1.5% | 1.6 |
| V111 | 6 | 0.4999 | 0.9% | 1.5% | 1.6 |
| M230L | 6 | 0.4999 | 0.9% | 1.5% | 1.6 |
| V245M | 60 | 0.5127 | 9.3% | 11.8% | 1.3 |
| E297A | 68 | 0.5344 | 10.6% | 13.2% | 1.3 |
| D177E | 150 | 0.5498 | 24.4% | 20.6% | 0.8 |
| E36 | 7 | 0.5547 | 1.1% | 1.5% | 1.4 |
| T69S | 7 | 0.5547 | 1.1% | 1.5% | 1.4 |
| Y188C | 7 | 0.5547 | 1.1% | 1.5% | 1.4 |
| I195 | 7 | 0.5547 | 1.1% | 1.5% | 1.4 |
| T215I | 7 | 0.5547 | 1.1% | 1.5% | 1.4 |
| M230 | 7 | 0.5547 | 1.1% | 1.5% | 1.4 |
| S251I | 7 | 0.5547 | 1.1% | 1.5% | 1.4 |
| T286P | 7 | 0.5547 | 1.1% | 1.5% | 1.4 |
| E6 | 33 | 0.5646 | 5.6% | 2.9% | 0.5 |
| V106I | 38 | 0.5920 | 5.9% | 7.4% | 1.2 |
| V245E | 96 | 0.5928 | 15.1% | 17.6% | 1.2 |
| G196E | 97 | 0.5956 | 15.2% | 17.6% | 1.2 |
| R172K | 8 | 0.6036 | 1.3% | 1.5% | 1.2 |
| N175 | 8 | 0.6036 | 1.3% | 1.5% | 1.2 |
| E204D | 8 | 0.6036 | 1.3% | 1.5% | 1.2 |
| K238T | 8 | 0.6036 | 1.3% | 1.5% | 1.2 |
| I50V | 9 | 0.6075 | 1.6% | 0.0% | 0.0 |
| A98G | 9 | 0.6075 | 1.6% | 0.0% | 0.0 |
| A288T | 9 | 0.6075 | 1.6% | 0.0% | 0.0 |
| K166R | 43 | 0.6097 | 7.2% | 4.4% | 0.6 |
| E28 | 10 | 0.6112 | 1.8% | 0.0% | 0.0 |
| I50 | 10 | 0.6112 | 1.8% | 0.0% | 0.0 |
| G190E | 10 | 0.6112 | 1.8% | 0.0% | 0.0 |
| D237 | 10 | 0.6112 | 1.8% | 0.0% | 0.0 |
| E169 | 47 | 0.6269 | 7.3% | 8.8% | 1.2 |
| V21 | 12 | 0.6295 | 1.8% | 2.9% | 1.6 |
| K173Q | 13 | 0.6424 | 2.0% | 2.9% | 1.5 |
| R211Q | 13 | 0.6424 | 2.0% | 2.9% | 1.5 |
| K238 | 13 | 0.6424 | 2.0% | 2.9% | 1.5 |
| L100I | 55 | 0.6491 | 8.6% | 10.3% | 1.2 |
| L100 | 55 | 0.6491 | 8.6% | 10.3% | 1.2 |
| I178L | 55 | 0.6491 | 8.6% | 10.3% | 1.2 |
| T39 | 59 | 0.6634 | 9.7% | 7.4% | 0.8 |
| I31L | 15 | 0.6728 | 2.3% | 2.9% | 1.3 |
| T69N | 15 | 0.6728 | 2.3% | 2.9% | 1.3 |
| P243 | 15 | 0.6728 | 2.3% | 2.9% | 1.3 |
| K103R | 16 | 0.6892 | 2.5% | 2.9% | 1.2 |
| E297 | 279 | 0.6992 | 44.3% | 47.1% | 1.1 |
| E138A | 17 | 0.7061 | 2.7% | 2.9% | 1.1 |
| E6D | 18 | 0.7090 | 3.0% | 1.5% | 0.5 |
| V276I | 20 | 0.7128 | 3.4% | 1.5% | 0.4 |
| K275 | 21 | 0.7172 | 3.6% | 1.5% | 0.4 |
| K11R | 22 | 0.7228 | 3.4% | 4.4% | 1.3 |
| K64R | 23 | 0.7296 | 3.6% | 4.4% | 1.2 |
| K64 | 23 | 0.7296 | 3.6% | 4.4% | 1.2 |
| S48 | 24 | 0.7373 | 3.8% | 4.4% | 1.2 |
| D121Y | 25 | 0.7458 | 3.9% | 4.4% | 1.1 |
| A272S | 27 | 0.7573 | 4.5% | 2.9% | 0.7 |
| I135T | 177 | 0.7772 | 28.5% | 26.5% | 0.9 |
| Q278 | 34 | 0.7787 | 5.4% | 5.9% | 1.1 |
| E291D | 37 | 0.7868 | 6.1% | 4.4% | 0.7 |
| E291 | 39 | 0.7891 | 6.5% | 4.4% | 0.7 |
| V245 | 237 | 0.7915 | 37.6% | 39.7% | 1.1 |
| E248D | 43 | 0.8009 | 6.8% | 7.4% | 1.1 |
| T165 | 49 | 0.8099 | 7.7% | 8.8% | 1.1 |
| K101E | 50 | 0.8119 | 7.9% | 8.8% | 1.1 |
| V179I | 61 | 0.8293 | 9.7% | 10.3% | 1.1 |
| E297K | 165 | 0.8845 | 26.5% | 25.0% | 0.9 |
| E53Q | 1 | 1.0000 | 0.2% | 0.0% | 0.0 |
| A62T | 1 | 1.0000 | 0.2% | 0.0% | 0.0 |
| K103T | 1 | 1.0000 | 0.2% | 0.0% | 0.0 |
| V108A | 1 | 1.0000 | 0.2% | 0.0% | 0.0 |
| S163C | 1 | 1.0000 | 0.2% | 0.0% | 0.0 |
| T215A | 1 | 1.0000 | 0.2% | 0.0% | 0.0 |
| T215C | 1 | 1.0000 | 0.2% | 0.0% | 0.0 |
| T215V | 1 | 1.0000 | 0.2% | 0.0% | 0.0 |
| K223E | 1 | 1.0000 | 0.2% | 0.0% | 0.0 |
| L234 | 1 | 1.0000 | 0.2% | 0.0% | 0.0 |
| Q242L | 1 | 1.0000 | 0.2% | 0.0% | 0.0 |
| Q242 | 1 | 1.0000 | 0.2% | 0.0% | 0.0 |
| I257V | 1 | 1.0000 | 0.2% | 0.0% | 0.0 |
| V292A | 1 | 1.0000 | 0.2% | 0.0% | 0.0 |
| T296A | 1 | 1.0000 | 0.2% | 0.0% | 0.0 |
| P4T | 2 | 1.0000 | 0.4% | 0.0% | 0.0 |
| I31V | 2 | 1.0000 | 0.4% | 0.0% | 0.0 |
| E36D | 2 | 1.0000 | 0.4% | 0.0% | 0.0 |
| E44D | 2 | 1.0000 | 0.4% | 0.0% | 0.0 |
| E44 | 2 | 1.0000 | 0.4% | 0.0% | 0.0 |
| D67G | 2 | 1.0000 | 0.4% | 0.0% | 0.0 |
| S163T | 2 | 1.0000 | 0.4% | 0.0% | 0.0 |
| K166Q | 2 | 1.0000 | 0.4% | 0.0% | 0.0 |
| G190V | 2 | 1.0000 | 0.4% | 0.0% | 0.0 |
| I195K | 2 | 1.0000 | 0.4% | 0.0% | 0.0 |
| T215S | 2 | 1.0000 | 0.4% | 0.0% | 0.0 |
| D250N | 2 | 1.0000 | 0.4% | 0.0% | 0.0 |
| K11T | 3 | 1.0000 | 0.5% | 0.0% | 0.0 |
| K70E | 3 | 1.0000 | 0.5% | 0.0% | 0.0 |
| K104N | 3 | 1.0000 | 0.5% | 0.0% | 0.0 |
| T139K | 3 | 1.0000 | 0.5% | 0.0% | 0.0 |
| N175Y | 3 | 1.0000 | 0.5% | 0.0% | 0.0 |
| E194D | 3 | 1.0000 | 0.5% | 0.0% | 0.0 |
| E40D | 4 | 1.0000 | 0.7% | 0.0% | 0.0 |
| V75M | 4 | 1.0000 | 0.7% | 0.0% | 0.0 |
| K101P | 4 | 1.0000 | 0.7% | 0.0% | 0.0 |
| S163 | 4 | 1.0000 | 0.7% | 0.0% | 0.0 |
| Y181V | 4 | 1.0000 | 0.7% | 0.0% | 0.0 |
| E194 | 4 | 1.0000 | 0.7% | 0.0% | 0.0 |
| E203D | 4 | 1.0000 | 0.7% | 0.0% | 0.0 |
| K238R | 4 | 1.0000 | 0.7% | 0.0% | 0.0 |
| K103S | 5 | 1.0000 | 0.9% | 0.0% | 0.0 |
| T165L | 5 | 1.0000 | 0.9% | 0.0% | 0.0 |
| G190C | 5 | 1.0000 | 0.9% | 0.0% | 0.0 |
| G196K | 5 | 1.0000 | 0.9% | 0.0% | 0.0 |
| L210F | 5 | 1.0000 | 0.9% | 0.0% | 0.0 |
| D237E | 5 | 1.0000 | 0.9% | 0.0% | 0.0 |
| D237N | 5 | 1.0000 | 0.9% | 0.0% | 0.0 |
| P243T | 5 | 1.0000 | 0.9% | 0.0% | 0.0 |
| K249R | 5 | 1.0000 | 0.9% | 0.0% | 0.0 |
| K275Q | 5 | 1.0000 | 0.9% | 0.0% | 0.0 |
| K32E | 6 | 1.0000 | 1.1% | 0.0% | 0.0 |
| V179E | 6 | 1.0000 | 1.1% | 0.0% | 0.0 |
| I244V | 6 | 1.0000 | 1.1% | 0.0% | 0.0 |
| I244 | 6 | 1.0000 | 1.1% | 0.0% | 0.0 |
| L301I | 6 | 1.0000 | 1.1% | 0.0% | 0.0 |
| E28K | 8 | 1.0000 | 1.4% | 0.0% | 0.0 |
| V189I | 8 | 1.0000 | 1.4% | 0.0% | 0.0 |
| V189 | 8 | 1.0000 | 1.4% | 0.0% | 0.0 |
| P4S | 9 | 1.0000 | 1.4% | 1.5% | 1.0 |
| R172 | 9 | 1.0000 | 1.4% | 1.5% | 1.0 |
| Q197E | 9 | 1.0000 | 1.4% | 1.5% | 1.0 |
| H208Y | 10 | 1.0000 | 1.6% | 1.5% | 0.9 |
| K249Q | 10 | 1.0000 | 1.6% | 1.5% | 0.9 |
| Q197K | 11 | 1.0000 | 1.8% | 1.5% | 0.8 |
| H208 | 11 | 1.0000 | 1.8% | 1.5% | 0.8 |
| Q278E | 11 | 1.0000 | 1.8% | 1.5% | 0.8 |
| E248N | 12 | 1.0000 | 2.0% | 1.5% | 0.7 |
| E6K | 14 | 1.0000 | 2.3% | 1.5% | 0.6 |
| D121H | 14 | 1.0000 | 2.3% | 1.5% | 0.6 |
| P4 | 15 | 1.0000 | 2.5% | 1.5% | 0.6 |
| K249 | 15 | 1.0000 | 2.5% | 1.5% | 0.6 |
| P225H | 16 | 1.0000 | 2.7% | 1.5% | 0.5 |
| P225 | 16 | 1.0000 | 2.7% | 1.5% | 0.5 |
| K275R | 16 | 1.0000 | 2.7% | 1.5% | 0.5 |
| I135L | 17 | 1.0000 | 2.9% | 1.5% | 0.5 |
| S48T | 19 | 1.0000 | 3.0% | 2.9% | 1.0 |
| I31 | 20 | 1.0000 | 3.2% | 2.9% | 0.9 |
| E204 | 21 | 1.0000 | 3.4% | 2.9% | 0.9 |
| V276 | 23 | 1.0000 | 3.8% | 2.9% | 0.8 |
| K101Q | 24 | 1.0000 | 3.9% | 2.9% | 0.7 |
| K11 | 31 | 1.0000 | 5.0% | 4.4% | 0.9 |
| Q197 | 32 | 1.0000 | 5.2% | 4.4% | 0.8 |
| P294T | 32 | 1.0000 | 5.2% | 4.4% | 0.8 |
| D121 | 41 | 1.0000 | 6.6% | 5.9% | 0.9 |
| E169D | 42 | 1.0000 | 6.8% | 5.9% | 0.9 |
| E248 | 56 | 1.0000 | 9.0% | 8.8% | 1.0 |
| P294 | 64 | 1.0000 | 10.2% | 10.3% | 1.0 |
| K101 | 94 | 1.0000 | 15.1% | 14.7% | 1.0 |
| G196 | 110 | 1.0000 | 17.6% | 17.6% | 1.0 |
| D177 | 160 | 1.0000 | 25.6% | 25.0% | 1.0 |
| R211K | 332 | 1.0000 | 53.0% | 52.9% | 1.0 |
| R211 | 392 | 1.0000 | 62.5% | 63.2% | 1.0 |
| R277K | 458 | 1.0000 | 73.1% | 73.5% | 1.0 |
| R277 | 458 | 1.0000 | 73.1% | 73.5% | 1.0 |
| K65R | 626 | 1.0000 | 100.0% | 100.0% | 1.0 |
| K65 | 626 | 1.0000 | 100.0% | 100.0% | 1.0 |

All references cited herein are incorporated by reference in their entireties.

The examples provided herein, both actual and prophetic, are merely embodiments of the present invention and are not intended to limit the invention in any way.

What is claimed is:

1. A method for determining that an HIV-1 is resistant to d4T, comprising detecting in a gene encoding reverse transcriptase of the HIV-1 the presence of a mutation at codon 65 in combination with a mutation at codon 40, 67, 69, 70, 75, 77, 116, 139, 169, 181, 190, 210, 215, 218, 219, 221, 224, or 227, wherein the presence of the mutations correlates with resistance to d4T, thereby determining that the HIV-1 is resistant to d4T.

2. The method of claim 1, wherein the mutation at codon 65 encodes arginine (R).

3. The method of claim 1, wherein the reverse transcriptase of the HIV-1 comprises a mutation at codon 40.

4. The method of claim 3, wherein the mutation at codon 40 encodes aspartic acid (D) or arginine (R).

5. The method of claim 1, wherein the reverse transcriptase of the HIV-1 comprises a mutation at codon 67.

6. The method of claim 1, wherein the mutation at codon 67 encodes glycine (G), asparagine (N), valine (V), or histidine (H).

7. The method of claim 1, wherein the reverse transcriptase of the HIV-1 comprises a mutation at codon 69.

8. The method of claim 7, wherein the mutation at codon 69 is a deletion of the amino acid present at codon 69 in a wild-type HIV-1 or encodes alanine (A), aspartic acid (D), isoleucine (I), asparagine (N), or serine (S).

9. The method of claim 1, wherein the reverse transcriptase of the HIV-1 comprises a mutation at codon 70.

10. The method of claim 9, wherein the mutation at codon 70 encodes cysteine, (C), glycine (G), histidine (H), asparagine (N), arginine (R), or threonine (T).

11. The method of claim 1, wherein the reverse transcriptase of the HIV-1 comprises a mutation at codon 75.

12. The method of claim 11, wherein the mutation at codon 75 encodes leucine (L), methionine (M), threonine (T), isoleucine (I), or alanine (A).

13. The method of claim 1, wherein the reverse transcriptase of the HIV-1 comprises a mutation at codon 77.

14. The method of claim 13, wherein the mutation at codon 77 encodes leucine (L).

15. The method of claim 1, wherein the reverse transcriptase of the HIV-1 comprises a mutation at codon 116.

16. The method of claim 15, wherein the mutation at codon 116 encodes proline (P), histidine (H), or tyrosine (Y).

17. The method of claim 1, wherein the reverse transcriptase of the HIV-1 comprises a mutation at codon 139.

18. The method of claim 17, wherein the mutation at codon 139 encodes alanine (A) or threonine (T).

19. The method of claim 1, wherein the reverse transcriptase of the HIV-1 comprises a mutation at codon 169.

20. The method of claim 19, wherein the mutation at codon 169 encodes alanine (A), aspartic acid (D), or lysine (K).

21. The method of claim 1, wherein the reverse transcriptase of the HIV-1 comprises a mutation at codon 181.

22. The method of claim 21, wherein the mutation at codon 181 encodes cysteine (C), glycine (G), isoleucine (I), leucine (L), or valine (V).

23. The method of claim 1, wherein the reverse transcriptase of the HIV-1 comprises a mutation at codon 190.

24. The method of claim 23, wherein the mutation at codon 190 encodes alanine (A), cysteine (C), glutamic acid (E), glutamine (Q), serine (S), or valine (V).

25. The method of claim 1, wherein the reverse transcriptase of the HIV-1 comprises a mutation at codon 210.

26. The method of claim 25, wherein the mutation at codon 210 encodes phenylalanine (F), methionine (M), or tryptophan (W).

27. The method of claim 1, wherein the reverse transcriptase of the HIV-1 comprises a mutation at codon 215.

28. The method of claim 27, wherein the mutation at codon 215 encodes alanine (A), cysteine (C), aspartic acid (D), glutamic acid (E), isoleucine (I), serine (S), valine (V), or tyrosine (Y).

29. The method of claim 1, wherein the reverse transcriptase of the HIV-1 comprises a mutation at codon 218.

30. The method of claim 29, wherein the mutation at codon 218 encodes glutamic acid (E).

31. The method of claim 1, wherein the reverse transcriptase of the HIV-1 comprises a mutation at codon 219.

32. The method of claim 31, wherein the mutation at codon 219 encodes histidine (H), threonine (T), arginine (R), glutamine (Q), or glutamic acid (E).

33. The method of claim 1, wherein the reverse transcriptase of the HIV-1 comprises a mutation at codon 221.

34. The method of claim 33, wherein the mutation at codon 221 encodes tyrosine (Y).

35. The method of claim 1, wherein the reverse transcriptase of the HIV-1 comprises a mutation at codon 224.

36. The method of claim 35, wherein the mutation at codon 224 encodes aspartic acid (D).

37. The method of claim 1, wherein the reverse transcriptase of the HIV-1 comprises a mutation at codon 227.

38. The method of claim 37, wherein the mutation at codon 227 encodes leucine (L).

39. A method for determining that an HIV-1 is susceptible to d4T, comprising detecting in a gene encoding reverse transcriptase of the HIV-1 the presence of a mutation at codon 65 in combination with a mutation at codon 135 or 184, wherein the presence of said mutations correlates with susceptibility to d4T, thereby determining that the HIV-1 is sensitive to d4T.

40. The method of claim 39, wherein the reverse transcriptase of the HIV-1 comprises a mutation at codon 135.

41. The method of claim 40, wherein the mutation at codon 135 encodes lysine (K), leucine (L), methionine (M), arginine (R), threonine (T), or valine (V).

42. The method of claim 39, wherein the reverse transcriptase of the HIV-1 comprises a mutation at codon 184.

43. The method of claim 42, wherein the mutation at codon 184 encodes isoleucine (I) or valine (V).

44. A computer-implemented method for determining that an HIV-1 is resistant to d4T, comprising inputting genetic information into a memory system of a computer, wherein the genetic information indicates that the HIV-1 has a gene encoding reverse transcriptase with a mutation at codon 65 in combination with a mutation at codon 40, 67, 69, 70, 75, 77, 116, 139, 169, 181, 190, 210, 215, 218, 219, 221, 224, or 227 of reverse transcriptase of the HIV-1, inputting a correlation between the presence of the mutations and resistance to d4T into the memory system of the computer, and determining that the HIV-1 is resistant to d4T.

45. The method of claim 44, further comprising displaying that the HIV-1 is resistant to d4T on a display of the computer.

46. The method of claim 44, further comprising printing that the HIV-1 is resistant to d4T.

47. A print-out indicating that an HIV-1 is resistant to d4T produced according to the method of claim 46.

48. A computer-readable medium comprising data indicating that an HIV-1 is resistant to d4T produced according to the method of claim 46.

49. A computer-implemented method for determining that an HIV-1 is susceptible to d4T, comprising inputting genetic information into a memory system of a computer, wherein the genetic information indicates that the HIV-1 has a gene encoding reverse transcriptase with a mutation at codon 65 in combination with a mutation at codon 135 or 184 of reverse transcriptase of the HIV-1, inputting a correlation between the presence of the mutations and resistance to d4T into the memory system of the computer, and determining that the HIV-1 is susceptible to d4T.

50. The method of claim 47, further comprising displaying that the HIV-1 is susceptible to d4T on a display of the computer.

51. The method of claim 47, further comprising printing that the HIV-1 is susceptible to d4T.

52. A print-out indicating that an HIV-1 is susceptible to d4T produced according to the method of claim 51.

53. A computer-readable medium comprising data indicating that an HIV-1 is susceptible to d4T produced according to the method of claim 51.


SEQUENCE LISTING (17 sequences)

SEQ ID NO: 1 - protein, 12 aa, HUMAN
  HIV-1 Partial Reverse Transcriptase Protein Sequence
KKKDSTKWRKLV

SEQ ID NO: 2 - protein, 11 aa, Human
  HIV-1 Partial Reverse Transcriptase Protein Sequence
KKKSGKWRKIT

SEQ ID NO: 3 - protein, 11 aa, Human
  HIV-1 Partial Reverse Transcriptase Protein Sequence
KKKSGRWRKLV

SEQ ID NO: 4 - protein, 11 aa, Human
  HIV-1 Partial Reverse Transcriptase Protein Sequence
KKKSGRWRKVV

SEQ ID NO: 5 - protein, 11 aa, Human
  HIV-1 Partial Reverse Transcriptase Protein Sequence
KKKSGKWRKLV

SEQ ID NO: 6 - protein, 11 aa, Human
  HIV-1 Partial Reverse Transcriptase Protein Sequence
KRKDGKWRKVV

SEQ ID NO: 7 - protein, 11 aa, Human
  HIV-1 Partial Reverse Transcriptase Protein Sequence
KKKSGKWRKIT

SEQ ID NO: 8 - protein, 11 aa, Human
  HIV-1 Partial Reverse Transcriptase Protein Sequence
KRKDGKWRKVV

SEQ ID NO: 9 - protein, 11 aa, Human
  HIV-1 Partial Reverse Transcriptase Protein Sequence
KKKDGKWRKLV

SEQ ID NO: 10 - protein, 11 aa, Human
  VARIANT 11: Xaa=Any Amino Acid; HIV-1 Partial Reverse Transcriptase Protein Sequence
KKKDGKWRKLX

SEQ ID NO: 11 - protein, 11 aa, Human
  HIV-1 Partial Reverse Transcriptase Protein Sequence
KKKDGKWRKLV

SEQ ID NO: 12 - protein, 11 aa, Human
  HIV-1 Partial Reverse Transcriptase Protein Sequence
KKKDGKWRKLV

SEQ ID NO: 13 - protein, 11 aa, Human
  HIV-1 Partial Reverse Transcriptase Protein Sequence
KKKDGKWRKLV

SEQ ID NO: 14 - protein, 11 aa, Human
  HIV-1 Partial Reverse Transcriptase Protein Sequence
KKKDGKWRKVV

SEQ ID NO: 15 - protein, 11 aa, Human
  VARIANT 3: Xaa=Any Amino Acid; HIV-1 Partial Reverse Transcriptase Protein Sequence
KKXDGKWRKVV

SEQ ID NO: 16 - protein, 11 aa, Human
  HIV-1 Partial Reverse Transcriptase Protein Sequence
KKKDSTWRKVV

SEQ ID NO: 17 - protein, 11 aa, Human
  HIV-1 Partial Reverse Transcriptase Protein Sequence
KKKDNTWRKLI